SEQUENCE LISTING (51 sequences)

SEQ ID NO: 1 - protein, 16 aa, Artificial Sequence
  Description of Artificial Sequence Synthetic peptide
SRVDGMVGDDPYNPYK

SEQ ID NO: 2 - protein, 15 aa, Artificial Sequence
  Description of Artificial Sequence Synthetic peptide
DTYERPRPGGRYRPG

SEQ ID NO: 3 - protein, 24 aa, Artificial Sequence
  Description of Artificial Sequence Synthetic peptide
RRLLRFSSQIHNNGQSDFRPKNGR

SEQ ID NO: 4 - protein, 14 aa, Artificial Sequence
  Description of Artificial Sequence Synthetic peptide
EDTSCDYGYHRRFA

SEQ ID NO: 5 - protein, 14 aa, Artificial Sequence
  Description of Artificial Sequence Synthetic peptide
EDTECEGDIQKNYE

SEQ ID NO: 6 - protein, 21 aa, Artificial Sequence
  Description of Artificial Sequence Synthetic peptide
DPYYIQASTYVQKMSMYNLRC

SEQ ID NO: 7 - protein, 21 aa, Artificial Sequence
  Description of Artificial Sequence Synthetic peptide
NAEMVQQTTYLEDRPMFMLQC

SEQ ID NO: 8 - protein, 14 aa, Artificial Sequence
  Description of Artificial Sequence Synthetic peptide
GSQYGPGRRRDPGA

SEQ ID NO: 9 - protein, 11 aa, Artificial Sequence
  Description of Artificial Sequence Synthetic peptide
WEWHSCHQHYH

SEQ ID NO: 10 - protein, 11 aa, Artificial Sequence
  Description of Artificial Sequence Synthetic peptide
WEWHSCHQHYH

SEQ ID NO: 11 - protein, 11 aa, Artificial Sequence
  Description of Artificial Sequence Synthetic peptide
WIWHDCHRHYH

SEQ ID NO: 12 - protein, 11 aa, Artificial Sequence
  Description of Artificial Sequence Synthetic peptide
WVWHECHGHYH

SEQ ID NO: 13 - protein, 11 aa, Artificial Sequence
  Description of Artificial Sequence Synthetic peptide
WVWHQCHRHYH

SEQ ID NO: 14 - protein, 17 aa, Artificial Sequence
  Description of Artificial Sequence Synthetic peptide
DIDCQWIDITDVKPGNY

SEQ ID NO: 15 - protein, 17 aa, Artificial Sequence
  Description of Artificial Sequence Synthetic peptide
DIDCQWIDITDVQPGNY

SEQ ID NO: 16 - protein, 17 aa, Artificial Sequence
  Description of Artificial Sequence Synthetic peptide
DIDCQWVDITDVPPGDY

SEQ ID NO: 17 - protein, 17 aa, Artificial Sequence
  Description of Artificial Sequence Synthetic peptide
DIDCQWIDITDVKPGNY

SEQ ID NO: 18 - protein, 17 aa, Artificial Sequence
  Description of Artificial Sequence Synthetic peptide
DIDCQWVDITDVGPGNY

SEQ ID NO: 19 - protein, 417 aa, Homo sapiens
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 20 - protein, 401 aa, Homo sapiens
ALVHCAPPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 21 - protein, 249 aa, Homo sapiens
DDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 22 - protein, 774 aa, Homo sapiens
MERPLCSHLCSCLAMLALLSPLSLAQYDSWPHYPEYFQQPAPEYHQPQAPANVAKIQLRLAGQKRKHSEGRVEVYYDGQWGTVCDDDFSIHAAHVVCRELGYVEAKSWTASSSYGKGEGPIWLDNLHCTGNEATLAACTSNGWGVTDCKHTEDVGVVCSDKRIPGFKFDNSLINQIENLNIQVEDIRIRAILSTYRKRTPVMEGYVEVKEGKTWKQICDKHWTAKNSRVVCGMFGFPGERTYNTKVYKMFASRRKQRYWPFSMDCTGTEAHISSCKLGPQVSLDPMKNVTCENGLPAVVSCVPGQVFSPDGPSRFRKAYKPEQPLVRLRGGAYIGEGRVEVLKNGEWGTVCDDKWDLVSASVVCRELGFGSAKEAVTGSRLGQGIGPIHLNEIQCTGNEKSIIDCKFNAESQGCNHEEDAGVRCNTPAMGLQKKLRLNGGRNPYEGRVEVLVERNGSLVWGMVCGQNWGIVEAMVVCRQLGLGFASNAFQETWYWHGDVNSNKVVMSGVKCSGTELSLAHCRHDGEDVACPQGGVQYGAGVACSETAPDLVLNAEMVQQTTYLEDRPMFMLQCAMEENCLSASAAQTDPTTGYRRLLRFSSQIHNNGQSDFRPKNGRHAWIWHDCHRHYHSMEVFTHYDLLNLNGTKVAEGHKASFCLEDTECEGDIQKNYECANFGDQGITMGCWDMYRHDIDCQWVDITDVPPGDYLFQVVINPNFEVAESDYSNNIMKCRSRYDGHRIWMYNCHIGGSFSEETEKKFEHFSGLLNNQLSPQ

SEQ ID NO: 23 - DNA, 1604 bp, Homo sapiens
  modified_base (1530)..(1530): n = a, c, g, t, unknown or other
gggcgtgatttgagccccgtttttattttctgtgagccacgtcctcctcgagggggtcaatctggccaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaacgtggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccactatgacctgcttgatgccaacacccagaggagagtggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattagaaggcaaagcaaaactcccaatggataaatcagtgcctggtgttctgaagtgggaaaaaatagactaacttcagtaggatttatgtattttgaaaaagagaacagaaaacaacaaaagaatttttgtttggactgttttcaataacaaagcacataactggattttgaacgcttaagtcaatcattacttggaaatttntaatgtttattatttacatcaactttgtgaattaacacagtgtttcaattctgtaatttcatatttgactcttt

SEQ ID NO: 24 - protein, 417 aa, Homo sapiens [sequence omitted]

SEQ ID NO: 25 - DNA, 1254 bp, Homo sapiens
atgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaacgtggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccactatgacctgcttgatgccaacacccagaggagagtggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattag

SEQ ID NO: 26 - protein, 417 aa, Homo sapiens [sequence omitted]

SEQ ID NO: 27 - DNA, 1780 bp, Homo sapiens
gggccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggggcgaacgcggacggccggctcatctggagtcaccgctggccgccccaggcccaccgcccgtcactggttccaagctggctactcgacatctagagcccgcgaagctgggccctcgcgcgcggagaaccagacagcgccgggagaagttcctgctctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccacttgtacctgcttgatgccaacacccagaggagatgggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattagaaggcaaagcaaaactcccaatggataaatcagtgcctggtgttctgaagtgggaaaaaatagactaacttcagtaggatttatgtattttgaaaaagagaacagaaaacaacaaaagaatttttgtttggactgttttcaataacaaagcacataactggattttgaacgcttaagtcatcattacttgggaaatttttaatgtttattatttacatcactttgtgaattaacacagtgtttcaattctgtaattacatatttgactctttcaaaaaaaaaaaaaaaaaaaaaaa

SEQ ID NO: 28 - protein, 417 aa, Homo sapiens
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAGRTRTAGSSGVTAGRPRPTARHWFQAGYSTSRAREAGPSRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHLYLLDANTQRRWAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 29 - DNA, 1935 bp, Homo sapiens
ccgcgccgctccccgttgccttccaggactgagaaaggggaaagggaagggtgccacgtccgagcagccgccttgactggggaagggtctgaatcccacccttggcattgcctggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaagctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccactatgacctgcttgatgccaacacccagaggagagtggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattagaaggcaaagcaaaactcccaatggataaatcagtgcctggtgttctgaagtgggaaaaaatagactaacttcagtaggatttatgtattttgaaaaagagaacagaaaacaacaaaagaatttttgtttggactgttttcaataacaaagcacataactggattttgaacgcttaagtcatcattacttgggaaatttttaatgtttattatttacatcactttgtgaattaacacagtgtttcaattctgtaattacatatttgactctttcaaagaaatccaaatttctcatgttccttttgaaattgtagtgcaaaatggtcagtattatctaaatgaatgagccaaaatgactttgaactgaaacttttctaaagtgctggaactttagtgaaacataataataatgggtttatacgacagcaacgga

SEQ ID NO: 30 - protein, 417 aa, Homo sapiens; identical to SEQ ID NO: 19

SEQ ID NO: 31 - DNA, 1322 bp, Homo sapiens
ggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaagctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccactatgacctgcttgatgccaacacccagaggagagtggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattagaaggcaaagcaaaactcccaatggataaatcagtgcctggtgttct

SEQ ID NO: 32 - protein, 417 aa, Homo sapiens; identical to SEQ ID NO: 19

SEQ ID NO: 33 - DNA, 1325 bp, Homo sapiens
ggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaagctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccactatgacctgcttgatgccaacacccagaggagagtggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattagaaggcaaagcaaaactcccaatggataaatcagtgcctggtgttctgaa

SEQ ID NO: 34 - protein, 407 aa, Homo sapiens
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCQWIDITDVKPGNYILKVSVNPSYLVPESDYTNNVVRCDIRYTGHHAYASGCTISPY

SEQ ID NO: 35 - DNA, 842 bp, Homo sapiens
gttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaagctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtaagtacccccaagtccgctggaagcacccgtgcacctggtccccagctatgtggcttcttctcgacgtggctgcctgggcgcggcgggccccggtcctcgcagatccgacccctccccacgcgcctgcagtggcagccctggaatccagtgcaaaccgcgcgtctggcccctcctgcttccttttcacattgctttgcagtcccgggggtccccagttctcttgctgtcctccgctccactctgcagtcccggtgggcgaagggtgaggagtaagggacctagaggggtagggagttggagcggggggcgccgggttgtttcactgctgcgcccgtcgcctgctgacgtttaggtctcccagacctggtg

SEQ ID NO: 36 - protein, 162 aa, Homo sapiens
FSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGDDPYNPYKYSDDNPYYNYYDTYERPRPGGRYRPGYGTGYFQYGLPDLV

SEQ ID NO: 37 - protein, 135 aa, Mus sp.
MEWSRVFIFLLSVTAGVHSQVQLQQSGAELVRPGTSVKVSCKASGYAFTYYLIEWVKQRPGQGLEWIGVINPGSGGTNYNEKFKGKATLTADKSSSTAYMQLSSLTSDDSAVYFCARNWMNFDYWGQGTTLTVSS

SEQ ID NO: 38 - protein, 116 aa, Artificial Sequence
  Description of Artificial Sequence Synthetic polypeptide
QVQLVQSGAELKKPGASVKVSCKASGYAFTYYLIEWVKQAPGQGLEWIGVINPGSGGTNYNEKFKGRATLTADKSTSTAYMELSSLRSEDSAVYFCARNWMNFDYWGQGTTVTVSS

SEQ ID NO: 39 - protein, 116 aa, Artificial Sequence
  Description of Artificial Sequence Synthetic polypeptide
QVQLVQSGAEVKKPGASVKVSCKASGYAFTYYLIEWVRQAPGQGLEWIGVINPGSGGTNYNEKFKGRATLTADKSTSTAYMELSSLRSEDTAVYFCARNWMNFDYWGQGTTVTVSS

SEQ ID NO: 40 - protein, 116 aa, Artificial Sequence
  Description of Artificial Sequence Synthetic polypeptide
QVQLVQSGAEVKKPGASVKVSCKASGYAFTYYLIEWVRQAPGQGLEWIGVINPGSGGTNYNEKFKGRATITADKSTSTAYMELSSLRSEDTAVYFCARNWMNFDYWGQGTTVTVSS

SEQ ID NO: 41 - protein, 116 aa, Artificial Sequence
  Description of Artificial Sequence Synthetic polypeptide
QVQLVQSGAEVKKPGASVKVSCKASGYAFTYYLIEWVRQAPGQGLEWIGVINPGSGGTNYNEKFKGRVTITADKSTSTAYMELSSLRSEDTAVYYCARNWMNFDYWGQGTTVTVSS

SEQ ID NO: 42 - protein, 132 aa, Mus sp.
MRCLAEFLGLLVLWIPGAIGDIVMTQAAPSVSVTPGESVSISCRSSKSLLHSNGNTYLYWFLQRPGQSPQFLIYRMSNLASGVPDRFSGSGSGTAFTLRISRVEAEDVGVYYCMQHLEYPYTFGGGTKLEIK

SEQ ID NO: 43 - protein, 112 aa, Artificial Sequence
  Description of Artificial Sequence Synthetic polypeptide
DIVMTQTPLSLSVTPGQPASISCRSSKSLLHSNGNTYLYWFLQKPGQSPQFLIYRMSNLASGVPDRFSGSGSGTAFTLKISRVEAEDVGVYYCMQHLEYPYTFGGGTKVEIK

SEQ ID NO: 44 - protein, 112 aa, Artificial Sequence
  Description of Artificial Sequence Synthetic polypeptide
DIVMTQTPLSLSVTPGQPASISCRSSKSLLHSNGNTYLYWFLQKPGQSPQFLIYRMSNLASGVPDRFSGSGSGTDFTLKISRVEAEDVGVYYCMQHLEYPYTFGGGTKVEIK

SEQ ID NO: 45 - protein, 112 aa, Artificial Sequence
  Description of Artificial Sequence Synthetic polypeptide
DIVMTQTPLSLSVTPGQPASISCRSSKSLLHSNGNTYLYWYLQKPGQSPQFLIYRMSNLASGVPDRFSGSGSGTDFTLKISRVEAEDVGVYYCMQHLEYPYTFGGGTKVEIK

SEQ ID NO: 46 - protein, 10 aa, Artificial Sequence
  Description of Artificial Sequence Synthetic peptide
GYAFTYYLIE

SEQ ID NO: 47 - protein, 17 aa, Artificial Sequence
  Description of Artificial Sequence Synthetic peptide
VINPGSGGTNYNEKFKG

SEQ ID NO: 48 - protein, 7 aa, Artificial Sequence
  Synthetic peptide
NWMNFDY

SEQ ID NO: 49 - protein, 16 aa, Artificial Sequence
  Description of Artificial Sequence Synthetic peptide
RSSKSLLHSNGNTYLY

SEQ ID NO: 50 - protein, 7 aa, Artificial Sequence
  Description of Artificial Sequence Synthetic peptide
RMSNLAS

SEQ ID NO: 51 - protein, 9 aa, Artificial Sequence
  Description of Artificial Sequence Synthetic peptide
MQHLEYPYT

(19) United States
(12) Patent Application Publication
(10) Pub. No.: US 2015/0361182 A1
(43) Pub. Date: Dec. 17, 2015

(54) METHODS FOR TREATING CARDIOVASCULAR DISEASES

(71) Applicant: Gilead Sciences, Inc., Foster City, CA (US)

(72) Inventors: Peidong Fan, San Mateo, CA (US); Jong Kang, Pleasanton, CA (US); Amanda Mikels-Vigdal, San Carlos, CA (US); Lina Yao, San Mateo, CA (US); Hongyan Zhong, Mountain View, CA (US)

(21) Appl. No.: 14/730,897

(22) Filed: Jun. 4, 2015

Related U.S. Application Data
(60) Provisional application No. 62/010,929, filed on Jun. 11, 2014.

(51) Int. Cl. C07K 16/40; G01N 33/573; A61L 31/10

(52) U.S. Cl. CPC C07K 16/40; A61L 31/10; G01N 33/573; C07K 2317/565; G01N 2800/325; G01N 2333/90638

(57) ABSTRACT

Provided herein are methods that relate to a novel therapeutic strategy for treatment of heart and/or cardiovascular diseases. The method includes administration of LOXL2 inhibitors for treating, preventing, or ameliorating at least one symptom associated with heart and/or cardiovascular diseases.

### CROSS REFERENCE TO RELATED APPLICATIONS

This application claims the benefit under 35 U.S.C. §119(e) of U.S. Provisional Application No. 62/010,929, filed Jun. 11, 2014, which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 1068-PC_{—}2015-06-04_sequence_listing.txt. The text file is 66.6 KB, was created on Jun. 1, 2015, and is being submitted electronically via EFS-Web.

### FIELD

The present application relates generally to the therapeutics and methods of using the same to treat or prevent diseases and conditions that affect the heart and/or cardiovascular system.

### BACKGROUND

Heart failure is the leading cause of morbidity and mortality. In the U.S. alone, approximately 500,000 people are diagnosed with heart failure each year, and a total of 5.7 million people are afflicted with heart failure. Under the current therapy, the one year mortality of heart failure is 30%, 5 year mortality is 50%, and 8 year mortality is 90%.

Accordingly, there is a need to develop new therapies.

### BRIEF SUMMARY

The invention generally relates to novel therapeutic strategies for treatment of heart and/or cardiovascular diseases comprising administration of LOXL2 inhibitors for treating, preventing, or ameliorating at least one symptom associated with heart failure and/or other cardiovascular diseases.

In various embodiments, a method for treating, preventing, or ameliorating at least one symptom associated with heart failure with preserved ejection fraction (HfpEF; diastolic heart failure (DHF), heart failure with reduced ejection fraction (HfrEF; systolic heart failure (SHF), cardiac arrhythmias and idiopathic dilated cardiomyopathy (IDCM), comprising: administering to a subject an effective amount of an inhibitor of active lysyl oxidase or lysyl oxidase-like protein is provided. In additional embodiments, a method for treating, preventing, or ameliorating at least one symptom associated with atrial fibrillation comprising: administering to a subject an effective amount of an inhibitor of active lysyl oxidase or lysyl oxidase-like protein is provided.

In various embodiments, a method for treating, preventing, or ameliorating at least one symptom associated with heart failure, comprising: administering to a subject an effective amount of an inhibitor of active lysyl oxidase or lysyl oxidase-like protein is provided.

In various other embodiments, a method for treating, preventing, or ameliorating at least one symptom associated with cardiac fibrosis, comprising: administering to a subject an effective amount of an inhibitor of active lysyl oxidase or lysyl oxidase-like protein is provided.

In various particular embodiments, a method for treating, preventing, or ameliorating at least one symptom associated with a cardiovascular injury selected from the group consisting of: IDCM, HFpEF, HFrEF, cardiac arrhythmias, and cardiac fibrosis, comprising: administering to a subject an effective amount of an inhibitor of active lysyl oxidase or lysyl oxidase-like protein is provided.

In certain embodiments, the cardiac arrhythmia is atrial fibrillation. In certain other embodiment, a method for treating, preventing, or ameliorating at least one symptom associated with atrial fibrillation (AF), comprising: administering to a subject an effective amount of an inhibitor of active lysyl oxidase or lysyl oxidase-like protein is provided.

In particular embodiments, ameliorating the one or more symptoms comprises reducing the extent of fibrosis, reducing myocardial remodeling, reducing myocardial stiffness during heart failure, reducing cardiac myofibroblast activation and/or improving systolic and diastolic heart function.

In certain embodiments, the survival of the subject is increased by at least 10 days, 1 month, 3 months, 6 months, 1 year, 1.5 years, 2 years, 3 years, 4 years, 5 years, 8 years, or 10 years.

In additional embodiments, the LOX or LOXL inhibitor is an antibody against LOX or LOXL, a small molecule inhibitor, siRNA, shRNA or an antisense polynucleotide against LOX or LOXL.

In further embodiments, the LOX or LOXL inhibitor is an antibody that specifically binds to a region of LOX or LOXL having an amino acid sequence selected from SEQ ID NOs: 1-22.

In particular embodiments, the LOX or LOXL inhibitor is parenterally administered to the subject.

In particular embodiments, the LOX or LOXL inhibitor is administered locally to a site of cardiovascular injury.

In some embodiments, the LOX or LOXL inhibitor is administered via a stent.

In additional embodiments, the LOX or LOXL inhibitor is coated on the stent.

In particular embodiments, the LOX or LOXL inhibitor is administered locally to a site of cardiovascular injury via a catheter.

In some embodiments, the LOX or LOXL inhibitor is administered prior to the onset or diagnosis of the cardiovascular injury.

In certain embodiments, the LOX or LOXL inhibitor is administered after the onset or diagnosis of the cardiovascular injury.

In particular embodiments, the LOX or LOXL inhibitor is administered at least 1, 2, 3, 5, or 10 hours after the onset or diagnosis of the cardiovascular injury or 1, 2, 3, 4, 5, 6, 7, 8, 9, 10, 11, 12, 13, or 14 days after the onset or diagnosis of the cardiovascular injury.

In various embodiments, an inhibitor of active lysyl oxidase or lysyl oxidase-like protein for use in treating, preventing, or ameliorating at least one symptom associated with a cardiovascular injury selected from the group consisting of: idiopathic dilated cardiomyopathy (IDCM), heart failure, cardiac arrhythmia, e.g., atrial fibrillation, and cardiac fibrosis is provided.

In various other embodiments, a composition comprising an inhibitor of lysyl oxidase, an inhibitor of a lysyl oxidase-like protein and a pharmaceutically acceptable carrier for use in treating, preventing, or ameliorating at least one symptom associated with a cardiovascular injury selected from the group consisting of: idiopathic dilated cardiomyopathy (IDCM), heart failure, cardiac arrhythmia, e.g., atrial fibrillation, and cardiac fibrosis is provided.

In certain embodiments, ameliorating the one or more symptoms comprises reducing the extent of fibrosis, reducing myocardial remodeling, reducing myocardial stiffness during heart failure, reducing cardiac myofibroblast activation and/or improving systolic and diastolic heart function.

In particular embodiments, the survival of the subject is increased by at least 10 days, 1 month, 3 months, 6 months, 1 year, 1.5 years, 2 years, 3 years, 4 years, 5 years, 8 years, or 10 years.

In additional embodiments, the LOX or LOXL inhibitor is an antibody against LOX or LOXL, a small molecule inhibitor, siRNA, shRNA or an antisense polynucleotide against LOX or LOXL.

In certain embodiments, the LOX or LOXL inhibitor is an antibody that specifically binds to a region of LOX or LOXL having an amino acid sequence selected from SEQ ID NOs: 1-22.

In various other embodiments, a method for diagnosing heart failure or atrial fibrillation in a subject is provided, comprising: contacting a serum sample obtained from an individual with an anti-LOXL2 antibody; detecting the binding of the anti-LOXL2 antibody to an anti-LOXL2 antibody/LOXL2 complex; wherein an increase in the level of an anti-LOXL2 antibody/LOXL2 complex compared to a reference sample indicates the presence of heart failure or atrial fibrillation in the subject.

In particular embodiments, the subject is suspected of having heart failure.

In certain embodiments, the heart failure is diastolic heart failure.

In further embodiments, the heart failure is systolic heart failure.

In some embodiments, the subject is suspected of having atrial fibrillation

In additional embodiments, anti-LOXL2 antibody binds to active LOXL2.

In some embodiments, the active LOXL2 is a mature form of LOXL2 after proteolytic processing of the preproprotein.

In particular embodiments, the anti-LOXL2 antibody is humanized or human.

In additional embodiments, the binding of the anti-LOXL2 antibody to the anti-LOXL2 antibody/LOXL2 complex is detected by enzyme-linked immunosorbent assays (ELISA).

In various other embodiments, a method for monitoring heart failure or atrial fibrillation in a subject is provided, comprising: contacting a serum sample obtained from an individual with an anti-LOXL2 antibody; detecting the binding of the anti-LOXL2 antibody to an anti-LOXL2 antibody/LOXL2 complex; wherein an increase in the level of an anti-LOXL2 antibody/LOXL2 complex compared to a reference sample indicates a worsening of heart failure or atrial fibrillation in the subject or wherein an decrease in the level of an anti-LOXL2 antibody/LOXL2 complex compared to a reference sample indicates an improvement of heart failure or atrial fibrillation in the subject.

In certain embodiments, the heart failure is diastolic heart failure.

In further embodiments, the heart failure is systolic heart failure.

In additional embodiments, anti-LOXL2 antibody binds to active LOXL2.

In some embodiments, the active LOXL2 is a mature form of LOXL2 after proteolytic processing of the preproprotein.

In particular embodiments, the anti-LOXL2 antibody is humanized or human.

In additional embodiments, the binding of the anti-LOXL2 antibody to the anti-LOXL2 antibody/LOXL2 complex is detected by enzyme-linked immunosorbent assays (ELISA).

In the various embodiments contemplated above and elsewhere herein, the LOXL2 inhibitor or the anti-LOXL2 antibody or antigen binding fragment thereof, comprises a heavy chain variable region comprising the amino acid sequence set forth as SEQ ID NO: 37, 38, 39, 40, or 41, and/or a light chain variable region comprising the amino acid sequence set forth as SEQ ID NO: 42, 43, 44, or 45, wherein the isolated antibody or antigen binding fragment thereof specifically binds a lysyl oxidase-like 2 (LOXL2) protein.

In particular embodiments, the LOXL2 inhibitor or the anti-LOXL2 antibody or antigen binding fragment thereof, comprises a heavy chain variable region comprising the amino acid sequence set forth as SEQ ID NO: 37, 38, 39, 40, or 41, and a light chain variable region comprising the amino acid sequence set forth as SEQ ID NO: 42, 43, 44, or 45.

In certain embodiments, the LOXL2 inhibitor or the anti-LOXL2 antibody or antigen binding fragment thereof, comprises the complementarity determining regions (CDRs), CDR1, CDR2, and CDR3, of a heavy chain variable region comprising the amino acid sequence set forth as SEQ ID NO: 37, 38, 39, 40, or 41, and the CDRs, CDR1, CDR2, and CDR3, of a light chain variable region comprising the amino acid sequence set forth as SEQ ID NO: 42, 43, 44, or 45, wherein the isolated antibody or antigen binding fragment thereof specifically binds a lysyl oxidase-like 2 (LOXL2) protein.

In additional embodiments, the LOXL2 inhibitor or the anti-LOXL2 antibody or antigen binding fragment thereof, comprises a heavy chain variable region comprises the CDR1-3 amino acid sequences set forth in SEQ ID NOs: 46-48. In further embodiments, the LOXL2 inhibitor or the anti-LOXL2 antibody or antigen binding fragment thereof, comprises a light chain variable region comprises the CDR1-3 amino acid sequences set forth in SEQ ID NOs: 49-51.

Other embodiments provide the uses of the LOXL2 inhibitors, including the anti-LOXL2 antibody or antigen binding fragment thereof, in the manufacture of a medicament for the treatment of a disease or condition that affect the heart and/or cardiovascular system. Also provided is a kit that includes a LOXL2 inhibitor. The kit may further comprise a label and/or instructions for use of the LOXL2 inhibitor, in treating a heart and/or cardiovascular disease in a human in need thereof. Further provided are articles of manufacture that include a LOXL2 inhibitor, and a container. In one embodiment, the container may be a vial, jar, ampoule, preloaded syringe, or an intravenous bag.

### BRIEF DESCRIPTION THE DRAWINGS

FIG. 1 shows LOXL2 serum protein levels (in pg/mL) measured using the VIDAS platform in serum samples from healthy subjects and patients with systolic heart failure (SHF).

### DETAILED DESCRIPTION

The following description sets forth exemplary methods, parameters and the like. It should be recognized, however, that such description is not intended as a limitation on the scope of the present disclosure but is instead provided as a description of exemplary embodiments.

In general, the present disclosure provides a method for treating or preventing or ameliorating at least one symptom associated with diseases and conditions that affect the heart and cardiovascular system, e.g., heart failure with reduced and preserved ejection fraction and atrial fibrillation.

The death in patients having heart failure is primarily caused by ventricular arrhythmias and/or pumping failure of the heart. Both ventricular arrhythmias and pumping failure may be related to the extent of cardiac fibrosis and adverse chamber remodeling (hypertrophy or chamber dilatation). Cardiac fibrosis, however, is an important determinant of cardiac dysfunction and abnormal chamber remodeling during heart failure.

Transaortic constriction (TAC) in mice causes pressure overload of the left ventricle of the heart, leading to hypertrophy and eventually heart failure in mice. It mimics the pressure effects of hypertension or aortic stenosis on the heart. Furthermore, the cardiac pathology caused by TAC—including hypertrophy, fibrosis, and chamber dilation—resembles that of cardiomyopathy caused by hypertension, aortic stenosis, or genetic mutations. Thus, TAC model serves as a suitable animal model to mimic human cardiomyopathy and heart failure. After TAC has been performed, the heart begins to show mild degrees of hypertrophy, fibrosis and diastolic dysfunction but without echocardiographic evidence of chamber dilation or contractile dysfunction. Cardiac hypertrophy, fibrosis and diastolic dysfunction continue to increase with time, and by the end of 4th week after TAC the heart displays echocardiographic evidence of cardiac systolic dysfunction with chamber dilatation and reduction of ejection fraction. The function of the pressure-overloaded hearts continues to deteriorate over time through the observation period of 10 to 12 weeks after the TAC procedure. The 14-day time point therefore marks the early stage of adverse cardiac remodeling and the transition from a compensated heart function to heart failure.

Heart failure is also associated with increased extracellular matrix (ECM) remodeling, marked myocardial fibrosis, and increased myocardial stiffness. Without wishing to be bound to any particular theory, it is contemplated that oxidative stress and hypoxia induced during heart failure and in other cardiovascular conditions, increases lysyl oxidase-like 2 (LOXL2) expression. LOXL2 catalyzes oxidative deamination of the lysine or hydroxylysine residues of collagen, leading to collagen cross-linking and myocardial stiffness. It is further contemplated that LOXL2 contributes to the activation of cardiac myofibroblasts in the development of myocardial fibrosis by increasing various cellular signaling pathways that results in the production of transforming growth factor-β (TGF-β), a key fibrogenic cytokine that sustains myofibroblast activation.

In various embodiments, therapeutic compositions and methods that target cardiovascular injuries including, but not limited to heart failure, idiopathic dilated cardiomyopathy (IDCM), cardiac arrhythmias, and cardiac fibrosis are provided.

In particular embodiments, therapeutic compositions and methods that target cardiac fibrosis to either reduce the extent of fibrosis, reduce myocardial remodeling, reduce myocardial stiffness during heart failure, atrial fibrillation, reduce cardiac myofibroblast activation, or that improve systolic and diastolic heart function are provided.

In certain embodiments, the therapeutic compositions comprise one or more agents that decrease or reduce the expression and/or activity of a LOX and/or LOXL enzyme.

### I. GENERAL DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. The headings provided herein are for convenience only and do not limit the invention in any way.

As used herein, “a” or “an” means “at least one” or “one or more.”

As used herein, the term “about” or “approximately” refers to a quantity, level, value, number, frequency, percentage, dimension, size, amount, weight, length, amino acid sequence, or polynucleotide sequence that varies by as much as 30, 25, 20, 25, 10, 9, 8, 7, 6, 5, 4, 3, 2, or 1% to a reference quantity, level, value, number, frequency, percentage, dimension, size, amount, weight, length, amino acid sequence, or polynucleotide sequence. In particular embodiments, the terms “about” or “approximately” when preceding a numerical value indicate the value plus or minus a range of 15%, 10%, 5%, or 1%.

The term “substantially” a quantity, level, value, number, frequency, percentage, dimension, size, amount, weight, length, amino acid sequence, or polynucleotide sequence at least about 60%, 65%, 75%, 80%85%, 90%, 91%, 92%, 93%, 94%, 95%, 96%, 97%, 98% or 99% identical to a reference quantity, level, value, number, frequency, percentage, dimension, size, amount, weight, length, amino acid sequence, or polynucleotide sequence.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “promoting,” “enhancing,” “stimulating,” or “increasing” generally refer to the ability of compositions contemplated herein to produce or cause a greater physiological response (i.e., measurable downstream effect), as compared to the response caused by either vehicle or a control molecule/composition. The physiological response may be increased by at least 5%, 10%, 20%, 30%, 40%, 50%, 60%, 70%, 80%, 90%, 100%, 125%, 150%, 175%, 200%, or greater compared to the response measured in normal, untreated, or control-treated subjects. An “increased” or “enhanced” response or property is typically “statistically significant”, and may include an increase that is 1.1, 1.2, 1.5, 2, 3, 4, 5, 6, 7, 8, 9, 10, 15, 20, 30, or more times (e.g., 500, 1000 times) (including all integers and decimal points in between and above 1, e.g., 1.5, 1.6, 1.7. 1.8, etc.) that produced by normal, untreated, or control-treated subjects.

As used herein, the terms “decrease” or “lower,” or “lessen,” or “reduce,” or “abate” refers generally to the ability of compositions contemplated to produce or cause a lesser physiological response (i.e., downstream effects), as compared to the response caused by either vehicle or a control molecule/composition. A “decrease” or “reduced” response is typically a “statistically significant” response, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by normal, untreated, or control-treated subject.

### II. LYSYL OXIDASE (LOX) AND LYSYL OXIDASE-LIKE (LOXL) PROTEINS

The expression of LOX and LOXL proteins varies in different diseases. This may be due to a number of reasons, such as the difference in tissue distribution, processing, domains, regulation of activity, as well as other differences between the proteins. For example, LOX and LOXL are implicated in fibrotic diseases as both LOX and LOXL are highly expressed in myo-fibroblasts around fibrotic areas (Kagen, Pathol. Res. Pract. 190:910-919 (1994); Murawaki et al., Hepatology 14:1167-1173 (1991); Siegel et al., Proc. Natl. Acad. Sci. USA 75:2945-2949 (1978); Jourdan Le-Saux et al., Biochem. Biophys. Res. Comm. 199:587-592 (1994); Kim et al., J. Cell Biochem. 72:181-188 (1999)).

Lysyl oxidase catalyzes oxidative deamination of peptidyl lysine and hydroxylysine residues in collagens, and peptidyl lysine residues in elastin. The resulting peptidyl aldehydes spontaneously condense and undergo oxidation reactions to form the lysine-derived covalent cross-links required for the normal structural integrity of the extracellular matrix. In the reaction of lysyl oxidase with its substrates, hydrogen peroxide (H₂O₂) and ammonium are released in quantities stoichiometric with the peptidyl aldehyde product. See, e.g., Kagan et al., J. Cell. Biochem. 88:660-72 (2003).

Lysyl oxidase is secreted into the extracellular environment where it is then processed by proteolytic cleavage to a functional 30 kDa enzyme and an 18 kDa propeptide. The 30 kDa lysyl oxidase is enzymatically active whereas the 50 kDa proenzyme is not. Procollagen C-proteinases process pro-lysyl oxidase to its active form and are products of the Bmp1, TII1 and TII2 genes. The localization of the enzyme is mainly extracellular, although processed lysyl oxidase also localizes intracellularly and nuclearly. Sequence coding for the propeptide is moderately (60-70%) conserved among LOX and the LOXL proteins, whereas the sequence coding for the C-terminal 30 kDa region of the proenzyme in which the active site is located is highly conserved (approximately 95%). See Kagan et al., J. Cell Biochem. 59:329-38 (1995).

Five different lysyl oxidases are known to exist in both humans and mice, LOX and four LOX related, or LOX-like proteins (LOXL1, LOXL2, LOXL3, LOXL4). LOX and the LOX-like proteins are referred to collectively as “LOX/LOXL” or “lysyl oxidase type enzymes” for the purposes of the present disclosure. The five forms of lysyl oxidases reside on five different chromosomes. These family members show some overlap in structure and function, but appear to have distinct functions as well. For example, although the main activity of LOX is the oxidation of specific lysine residues in collagen and elastin outside of the cell, it may also act intracellularly, where it may regulate gene expression. In addition, LOX induces chemotaxis of monocytes, fibroblasts and smooth muscle cells. Further, a deletion of LOX in knockout mice appears to be lethal at parturition (Hornstra et al., J. Biol. Chem. 278:14387-14393 (2003)), whereas LOXL deficiency causes no severe developmental phenotype (Bronson et al., Neurosci. Lett. 390:118-122 (2005)).

The main activity of LOX is the oxidation of specific lysine residues in collagen and elastin outside of the cell, however, it may also act intracellularly, where it may regulate gene expression (Li et al., Proc. Natl. Acad. Sci. USA 94:12817-12822 (1997), Giampuzzi et al., J. Biol. Chem. 275:36341-36349 (2000)). In addition, LOX induces chemotaxis of monocytes, fibroblasts and smooth muscle cells (Lazarus et al., Matrix Biol. 14:727-731 (1995), Nelson et al., Proc. Soc. Exp. Biol. Med. 188:346-352 (1988)). LOX itself is induced by a number of growth factors and steroids such as TGF-β, TNF-α and interferon (Csiszar, Prog. Nucl. Acid Res. 70:1-32 (2001)). Recent studies have attributed other roles to LOX in diverse biological functions such as developmental regulation, tumor suppression, cell motility, and cellular senescence. The diverse role of LOX, and its recently discovered amino oxidase family, LOX-like (LOXL), may play important roles with their intracellular and extracellular localization.

As used herein, the term “lysyl oxidase” refers to an enzyme that catalyzes the following reaction: peptidyl-L-lysyl-peptide+O₂+H₂O peptidyl-allysyl-peptide+NH₃+H₂O₂. Other synonyms for lysyl oxidase (EC 1.4.3.13) include protein-lysine 6-oxidase and protein-L-lysine: oxygen 6-oxidoreductase (deaminating). See, e.g., Harris et al., Biochim. Biophys. Acta 341:332-44 (1974); Rayton et al., J. Biol. Chem. 254:621-26 (1979); Stassen, Biophys. Acta 438:49-60 (1976). A copper-containing quinoprotein with a lysyl adduct of tyrosyl quinone at its active center, LOX catalyzes the oxidation of peptidyl lysine to result in the formation of peptidyl alpha-aminoadipic-delta-semialdehyde. Once formed, this semialdehyde can spontaneously condense with neighboring aldehydes or with other lysyl groups to form intra- and interchain cross-links. See, e.g., Rucker et al., Am. J. Clin. Nutr. 67:996S-1002S (1998).

An example of lysyl oxidase or lysyl oxidase-like protein include the enzyme having an amino acid sequence substantially identical to a polypeptide expressed or translated from one of the following sequences: EMBL/GenBank accession numbers: M94054 (SEQ ID NO: 23); AAA59525.1 (SEQ ID NO: 24); 545875 (SEQ ID NO: 25); AAB23549.1 (SEQ ID NO: 26); 578694 (SEQ ID NO: 27); AAB21243.1 (SEQ ID NO: 28); AF03929 I (SEQ ID NO: 29); AAD02130.1 (SEQ ID NO: 30); BC074820 (SEQ ID NO: 31); AAH74820.1 (SEQ ID NO: 32); BC074872 (SEQ ID NO: 33); AAH74872.1 (SEQ ID NO: 34); M84150 (SEQ ID NO: 35); AAA59541.1 (SEQ ID NO: 36). One embodiment of LOX is human lysyl oxidase (hLOX) preproprotein having an amino acid sequence (SEQ ID NO: 19), a secreted hLOX after cleavage of the signal peptide such as SEQ ID NO: 20 or a mature hLOX after proteolytic processing such as SEQ ID NO: 21. In one embodiment, the LOXL is human LOXL2, e.g., SEQ ID NO: 22.

LOX has highly conserved protein domains, conserved in several species including human, mouse, rat, chicken, fish and Drosophila. The human LOX family has a highly conserved C-terminal region containing the 205 amino acid LOX catalytic domain. The conserved region contains the copper binding (Cu), conserved cytokine receptor like domain (CRL), and the lysyl-tyrosylquinone cofactor site (LTQ). The predicted extracellular signal sequences are represented by the hatched boxes (See FIG. 7 of U.S. Provisional Application No. 60/963,249, which is incorporated herein by reference in its entirety). Twelve cysteine residues are also similarly conserved, wherein two of them reside within the prepropeptide region and ten are in the catalytically active processed form of LOX (Csiszar, Prog. Nucl. Acid Res. 70:1-32 (2001)). The conserved region also includes a fibronectin binding domain.

The prepropeptide region of LOX contains the signal peptide, and is cleaved, the cleavage site predicted to be between Cys21-Ala22, to generate a signal sequence peptide and a 48 kDa amino acid propeptide form of LOX, which is still inactive. The propeptide is N-glycosylated during passage through the Golgi that is secreted into the extracellular environment where the proenzyme, or propeptide, is cleaved between Gly168-Asp169 by a metalloendoprotease, a procollagen C-proteinase, which are products of the Bmp1, TII1 and TII2 genes. BMP I (bone morphogenetic protein I) is a procollagen C-proteinase that processes the propeptide to yield a functional 30 kDa enzyme and an 18 kDa propeptide. The sequence coding for the propeptide is moderately (60-70%) conserved, whereas the sequence coding for the C-terminal 30 kDa region of the proenzyme in which the active site is located is highly conserved (approximately 95%). (Kagan and Li, J. Cell. Biochem. 88:660-672 (2003); Kagan et al., J. Cell Biochem. 59:329-38 (1995)). The N-glycosyl units are also subsequently removed. LOX occurs in unprocessed and/or processed (mature) forms. The mature form of LOX is typically active although, in some embodiments, unprocessed LOX is also active.

Particular examples of a LOXL enzyme or protein are described in Molnar et al., Biochim Biophys Acta. 1647:220-24 (2003); Csiszar, Prog. Nucl. Acid Res. 70:1-32 (2001); and in WO01/83702 published on Nov. 8, 2001, all of which are herein incorporated by reference in their entirety. (It is noted that in these 3 publications, “LOXL1” was referred to as “LOXL” whereas in the present invention “LOXL” is used to refer to a lysyl oxidase-like proteins in general, not just LOXL1.) These enzymes include LOXL1, encoded by mRNA deposited at GenBank/EMBL BC015090; AAH15090.1; LOXL2, encoded by mRNA deposited at GenBank/EMBL U89942; LOXL3, encoded by mRNA deposited at GenBank/EMBL AF282619; AAK51671.1; and LOXL4, encoded by mRNA deposited at GenBank/EMBL AF338441; AAK71934.1.

Similar potential signal peptides as those described above for LOX have been predicted at the amino terminus of LOXL, LOXL2, LOXL3, and LOXL4. The predicted signal cleavage sites are between Gly25-Gln26 for LOXL, between Ala25-Gln26, for LOXL2, and between Gly25-Ser26 for LOXL3. The consensus for BMP-1 cleavage in pro-collagens and pro-LOX is between Ala/Gly-Asp, and often followed by an acidic or charged residue. A potential cleavage site to generate active LOXL is Gly303-Asp304, however, it is then followed by an atypical Pro. LOXL3 also has a potential cleavage site at Gly447-Asp448, which is followed by an Asp, processing at this site may yield an active peptide of similar size to active LOX. A potential cleavage site of BMP-I was also identified within LOXL4, at residues Ala569-Asp570 (Kim et al., J. Biol. Chem. 278:52071-52074 (2003)). LOXL2 may also be proteolytically cleaved analogously to the other members of the LOXL family and secreted (Akiri et al., Cancer Res. 63:1657-1666 (2003)).

The terms “LOX” and “LOXL” also encompass functional fragments or derivatives that substantially retain enzymatic activity catalyzing the deamination of lysyl residues. Typically, a functional fragment or derivative retains at least 50% of 60%, 70%, 80%, 90%, 95%, 99% or 100% of its lysyl oxidation activity. It is also intended that a LOX or a LOXL2 protein can include conservative amino acid substitutions that do not substantially alter its activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity. See, e.g., Watson, et al., Molecular Biology of the Gene, 4th Edition, 1987, The Benjamin/Cummings Pub. Co., p. 224. Conservative and non-conservative amino acid substitutions have been described above.

A feature not known to be common amongst the LOX and LOXL proteins is the scavenger receptor cysteine rich (SRCR) domains. LOX and LOXL lack SRCR domains, whereas LOXL2, LOXL3, and LOXL4 each have four SRCR domains at the N-terminus SRCR domains are found in secreted, transmembrane, or extracellular matrix proteins. SRCR domains are also known to mediate ligand binding in a number of secreted and receptor proteins (Hoheneste et al., Nat. Struct. Biol. 6:228-232 (1999); Sasaki et al., EMBO J. 17:1606-1613 (1998)). Another domain unique to LOXL is the presence of a proline rich domain (Molnar et al., Biochimica Biophsyica Acta 1647:220-224 (2003)).

### III. Epithelial—Mesenchymal Transition

Epithelial-to-Mesenchymal Transition (EMT) refers to the process whereby a cell with a gene expression/phenotype characteristic of epithelial cell (i.e., expressing specific proteins, factors, and molecules) changes or alters the genes or their level of expression which results in a change in the phenotype of the cell as exhibited by the alteration or change in the genes expressed.

Epithelial and mesenchymal cells represent distinct lineages, each with a unique gene expression profile that imparts attributes specific to each cell type. Conversion of an epithelial cell into a mesenchymal cell requires alterations in morphology, cellular architecture, adhesion, and/or migration capacity. Molecular and morphologic features indicative of EMT correlate with fibrosis.

### IV. Agents that Decrease LOX and LOXL Expression and/or Activity

In various embodiments, methods of treating or preventing or ameliorating one or more symptoms associated with heart failure, idiopathic dilated cardiomyopathy (IDCM), and cardiac fibrosis comprising administering one or more agents, e.g., therapeutic agents, that reduces LOX/LOXL expression and/or activity are provided. As used herein, the terms “agent” and “therapeutic agent” may be used interchangeable in particular embodiments. Agents contemplated herein include, but are not limited to small molecules; inhibitory polynucleotides including but not limited to siRNA, shRNA, miRNA, piRNA, and antisense oligonucleotides; and inhibitory polypeptides, including but not limited to antibodies and antigen binding fragments thereof.

In particular embodiments, methods of reducing the extent of fibrosis, myocardial remodeling, myocardial stiffness during heart failure, cardiac myofibroblast activation comprising administering one or more agents that reduces LOX/LOXL expression and/or activity are provided.

In particular embodiments, methods of improving systolic and diastolic heart function comprising administering one or more agents that reduces LOX/LOXL expression and/or activity are provided.

Agents that reduce, decrease, or inhibit the activity of LOX/LOXL enzymes include, but are not limited to, small molecule-, polynucleotide-, and polypeptide-based inhibitors and antagonists of LOX, LOXL1, LOXL2, LOXL3, and LOXL4. Such agents are referred to as therapeutic agents. The agents can be selected by using a variety of screening assays. In one embodiment, inhibitors can be identified by determining if a test compound binds to a lysyl oxidase-type enzyme; wherein, if binding has occurred, the compound is a candidate modulator. Optionally, additional tests can be carried out on such a candidate modulator. Alternatively, a candidate compound can be contacted with a lysyl oxidase-type enzyme, and a biological activity of the lysyl oxidase-type enzyme assayed; a compound that alters the biological activity of the lysyl oxidase-type enzyme is a modulator of a lysyl oxidase-type enzyme. Generally, a compound that reduces a biological activity of a lysyl oxidase-type enzyme is an inhibitor of the enzyme.

In one embodiment, the LOX/LOXL inhibitor is a LOXL2 inhibitor.

Methods of identifying modulators of the activity of lysyl oxidase-type enzymes include incubating a candidate compound in a cell culture containing one or more lysyl oxidase-type enzymes and assaying one or more biological activities or characteristics of the cells. Compounds that alter the biological activity or characteristic of the cells in the culture are potential modulators of the activity of a lysyl oxidase-type enzyme. Biological activities that can be assayed include, for example, lysine oxidation, peroxide production, ammonia production, levels of lysyl oxidase-type enzyme, levels of mRNA encoding a lysyl oxidase-type enzyme, and/or one or more functions specific to a lysyl oxidase-type enzyme. In additional embodiments of the aforementioned assay, in the absence of contact with the candidate compound, the one or more biological activities or cell characteristics are correlated with levels or activity of one or more lysyl oxidase-type enzymes. For example, the biological activity can be a cellular function such as migration, chemotaxis, epithelial-to-mesenchymal transition, or mesenchymal-to-epithelial transition, and the change is detected by comparison with one or more control or reference sample(s). For example, negative control samples can include a culture with decreased levels of a lysyl oxidase-type enzyme to which the candidate compound is added; or a culture with the same amount of lysyl oxidase-type enzyme as the test culture, but without addition of candidate compound. In some embodiments, separate cultures containing different levels of a lysyl oxidase-type enzyme are contacted with a candidate compound. If a change in biological activity is observed, and if the change is greater in the culture having higher levels of lysyl oxidase-type enzyme, the compound is identified as a modulator of the activity of a lysyl oxidase-type enzyme. Determination of whether the compound is an activator or an inhibitor of a lysyl oxidase-type enzyme may be apparent from the phenotype induced by the compound, or may require further assay, such as a test of the effect of the compound on the enzymatic activity of one or more lysyl oxidase-type enzymes.

Methods for obtaining lysyl oxidase-type enzymes, either biochemically or recombinantly, as well as methods for cell culture and enzymatic assay to identify modulators of the activity of lysyl oxidase-type enzymes as described above, are known in the art.

The structure of the lysyl oxidase-type enzymes can be investigated to guide the selection of agents such as, for example, small molecules, peptides, peptide mimetics and antibodies. Structural properties of a lysyl oxidase-type enzyme can help to identify natural or synthetic molecules that bind to, or function as a ligand, substrate, binding partner or the receptor of, the lysyl oxidase-type enzyme. See, e.g., Engleman (1997) J. Clin. Invest. 99:2284-2292. For example, folding simulations and computer redesign of structural motifs of lysyl oxidase-type enzymes can be performed using appropriate computer programs. Olszewski (1996) Proteins 25:286-299; Hoffman (1995) Comput. Appl. Biosci. 11:675-679. Computer modeling of protein folding can be used for the conformational and energetic analysis of detailed peptide and protein structure. Monge (1995) J. Mol. Biol. 247:995-1012; Renouf (1995) Adv. Exp. Med. Biol. 376:37-45. Appropriate programs can be used for the identification of sites, on lysyl oxidase-type enzymes, that interact with ligands and binding partners, using computer assisted searches for complementary peptide sequences. Fassina (1994) Immunomethods 5:114-120. Additional systems for the design of protein and peptides are described, for example in Berry (1994) Biochem. Soc. Trans. 22:1033-1036; Wodak (1987), Ann. N.Y. Acad. Sci. 501:1-13; and Pabo (1986) Biochemistry 25:5987-5991. The results obtained from the above-described structural analyses can be used for, e.g., the preparation of organic molecules, peptides and peptide mimetics that function as modulators of the activity of one or more lysyl oxidase-type enzymes.

An inhibitor of a lysyl oxidase-type enzyme can be a competitive inhibitor, an uncompetitive inhibitor, a mixed inhibitor or a non-competitive inhibitor. Competitive inhibitors often bear a structural similarity to substrate, usually bind to the active site, and are more effective at lower substrate concentrations. The apparent K_{M} is increased in the presence of a competitive inhibitor. Uncompetitive inhibitors generally bind to the enzyme-substrate complex or to a site that becomes available after substrate is bound at the active site and may distort the active site. Both the apparent K_{M} and the Vₘₐₓ are decreased in the presence of an uncompetitive inhibitor, and substrate concentration has little or no effect on inhibition. Mixed inhibitors are capable of binding both to free enzyme and to the enzyme-substrate complex and thus affect both substrate binding and catalytic activity. Non-competitive inhibition is a special case of mixed inhibition in which the inhibitor binds enzyme and enzyme-substrate complex with equal avidity, and inhibition is not affected by substrate concentration. Non-competitive inhibitors generally bind to enzyme at a region outside the active site. For additional details on enzyme inhibition see, for example, Voet et al. (2008) supra. For enzymes such as the lysyl oxidase-type enzymes, whose natural substrates (e.g., collagen, elastin) are normally present in vast excess in vivo (compared to the concentration of any inhibitor that can be achieved in vivo), noncompetitive inhibitors are advantageous, since inhibition is independent of substrate concentration.

The enzymatic activity of a lysyl oxidase-type enzyme can be assayed by a number of different methods. For example, lysyl oxidase enzymatic activity can be assessed by detecting and/or quantitating production of hydrogen peroxide, ammonium ion, and/or aldehyde, by assaying lysine oxidation and/or collagen crosslinking, or by measuring cellular invasive capacity, cell adhesion, cell growth or metastatic growth. See, for example, Trackman et al. (1981) Anal. Biochem. 113:336-342; Kagan et al. (1982) Meth. Enzymol. 82A:637-649; Palamakumbura et al. (2002) Anal. Biochem. 300:245-251; Albini et al. (1987) Cancer Res. 47:3239-3245; Kamath et al. (2001) Cancer Res. 61:5933-5940; U.S. Pat. No. 4,997,854 and U.S. patent application publication No. 2004/0248871.

### Small Molecules

In particular embodiments, the agent comprises one or more small molecules that reduce, decrease, or inhibit the activity of LOX/LOXL enzymes. A “small molecule” refers to an agent that has a molecular weight of less than about 5 kD, less than about 4 kD, less than about 3 kD, less than about 2 kD, less than about 1 kD, or less than about 0.5 kD. Small molecules include, but are not limited to: nucleic acids, peptidomimetics, peptoids, carbohydrates, lipids or other organic or inorganic molecules. Libraries of chemical and/or biological mixtures, such as fungal, bacterial, or algal extracts, are known in the art and can be used as a source of small molecules in certain embodiments. In particular embodiments, the small molecule has a molecular weight of less than 10,000 daltons, for example, less than 8000, 6000, 4000, 2000 daltons, e.g., between 50-1500, 500-1500, 200-2000, 500-5000 daltons.

In particular embodiments, the small molecule has a molecular weight of less than 10,000 daltons, for example, less than 8000, 6000, 4000, 2000 daltons, e.g., between 50-1500, 500-1500, 200-2000, 500-5000 daltons. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994). Libraries of compounds may be presented in solution (Houghten et al., 1992) or on beads (Lam et al., 1991), on chips (Fodor et al., 1993), bacteria, spores (Ladner et al., U.S. Pat. No. 5,223,409, 1993), plasmids (Cull et al., 1992) or on phage (Cwirla et al., 1990; Devlin et al., 1990; Felici et al., 1991; Ladner et al., U.S. Pat. No. 5,223,409, 1993; Scott and Smith, 1990).

Libraries useful for the purposes of the invention include, but are not limited to, (1) chemical libraries, (2) natural product libraries, and (3) combinatorial libraries comprised of random peptides, oligonucleotides and/or organic molecules.

Chemical libraries consist of structural analogs of known compounds or compounds that are identified as “hits” or “leads” via natural product screening. Natural product libraries are derived from collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see, Cane, D. E., et al., (1998) Science 282:63-68. Combinatorial libraries are composed of large numbers of peptides, oligonucleotides or organic compounds as a mixture. They are relatively easy to prepare by traditional automated synthesis methods, PCR, cloning or proprietary synthetic methods.

### Antibodies

In various embodiments, the one or more agents that reduce LOXL2 expression and/or activity comprise antibodies and antigen binding fragments useful in such methods are those, for example, that specifically bind LOX or LOXL2. See, e.g., U.S. Patent Applications, 20090053224 and 20090104201, the disclosures of which, including all anti-LOX, anti-LOXL1, anti-LOXL2, anti-LOXL3, and anti-LOXL4 antibody sequences (including CDR, heavy chain and light chain sequences), methods of making the antibodies, and antibody variants, are herein incorporated by reference in their entireties.

As used herein, the term “antibody” means an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′, F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or an isolated V_{H} or V_{L} region comprising only three of the six CDRs specific for an antigen) has the ability to recognize and bind antigen, although generally at a lower affinity than does the entire Fv fragment.

The “Fab” fragment also contains, in addition to heavy and light chain variable regions, the constant domain of the light chain and the first constant domain (CH1) of the heavy chain. Fab fragments were originally observed following papain digestion of an antibody. Fab′ fragments differ from Fab fragments in that F(ab′) fragments contain several additional residues at the carboxy terminus of the heavy chain CH1 domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain, thereby creating two antigen-binding sites. Diabodies are additionally described, for example, in EP 404,097; WO 93/11161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

In some embodiments, an antibody is a humanized antibody or a human antibody. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies can also comprise residues that are found neither in the recipient antibody nor in the imported CDR or framework sequences. In certain embodiments, a humanized antibody comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies can also include immunoglobulin fragments, such as Fv, Fab, Fab′, F(ab′)₂ or other antigen-binding subsequences of antibodies.

The humanized antibody can also comprise at least a portion of an immunoglobulin constant region (Fc), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies are known in the art. Generally, a humanized antibody has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies include chimeric antibodies (U.S. Pat. No. 4,816,567), wherein substantially less than an intact human variable domain has been substituted by the corresponding sequence from a non-human species. In certain embodiments, humanized antibodies are human antibodies in which some CDR residues and optionally some framework region residues are substituted by residues from analogous sites in rodent antibodies (e.g., murine monoclonal antibodies).

Human antibodies can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; and Lonberg et al. (1995) Intern. Rev. Immunol. 13:65-93.

Antibodies can be affinity matured using known selection and/or mutagenesis methods as described above. In some embodiments, affinity matured antibodies have an affinity which is five times or more, ten times or more, twenty times or more, or thirty times or more than that of the starting antibody (generally murine, rabbit, chicken, humanized or human) from which the matured antibody is prepared.

An antibody can also be a bispecific antibody. Bispecific antibodies are monoclonal, and may be human or humanized antibodies that have binding specificities for at least two different antigens. In the present case, the two different binding specificities can be directed to two different lysyl oxidase-type enzymes, or to two different epitopes on a single lysyl oxidase-type enzyme.

An antibody as disclosed herein can also be an immunoconjugate. Such immunoconjugates comprise an antibody (e.g., to a lysyl oxidase-type enzyme) conjugated to a second molecule, such as a reporter An immunoconjugate can also comprise an antibody conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

An antibody that “specifically binds to” or is “specific for” a particular polypeptide or an epitope on a particular polypeptide is one that binds to that particular polypeptide or epitope without substantially binding to any other polypeptide or polypeptide epitope. In some embodiments, an antibody of the present disclosure specifically binds to its target with a dissociation constant (Kₐ) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM; in the form of monoclonal antibody, scFv, Fab, or other form of antibody measured at a temperature of about 4° C., 25° C., 37° C., or 42° C.

In certain embodiments, an antibody of the present disclosure binds to one or more processing sites (e.g., sites of proteolytic cleavage) in a lysyl oxidase-type enzyme, thereby effectively blocking processing of the proenzyme or preproenzyme to the catalytically active enzyme, thereby reducing the activity of the lysyl oxidase-type enzyme.

In certain embodiments, an antibody according to the present disclosure binds to human LOXL2 with a greater binding affinity, for example, at least 10 times, at least 100 times, or even at least 1000 times greater than its binding affinity to other lysyl oxidase-type enzymes, e.g., LOX, LOXL1, LOXL3, and LOXL4.

In certain embodiments, an antibody according to the present disclosure is a non-competitive inhibitor of the catalytic activity of a lysyl oxidase-type enzyme. In certain embodiments, an antibody according to the present disclosure binds outside the catalytic domain of a lysyl oxidase-type enzyme. In certain embodiments, an antibody according to the present disclosure binds to the SRCR4 domain of LOXL2. In certain embodiments, an anti-LOXL2 antibody that binds to the SRCR4 domain of LOXL2 and functions as a non-competitive inhibitor is the AB0023 antibody, described in co-owned U.S. Patent Application Publications No. US 2009/0053224 and US 2009/0104201, the disclosures of which, including all anti-LOX, anti-LOXL1, anti-LOXL2, anti-LOXL3, and anti-LOXL4 antibody sequences (including CDR, heavy chain and light chain sequences), methods of making the antibodies, and antibody variants, are herein incorporated by reference in their entireties. In certain embodiments, an anti-LOXL2 antibody that binds to the SRCR4 domain of LOXL2 and functions as a non-competitive inhibitor is the AB0024 antibody (a human version of the AB0023 antibody), described in co-owned U.S. Patent Application Publications No. US 2009/0053224 and US 2009/0104201. Additional exemplified anti-LOXL2 antibody or antigen binding fragment thereof may be found in U.S. patent application publication nos. 2012/0309020, 2013/0324705, 2014/0079707, and 2011/0200606; each of which is incorporated herein by reference in the entirety. In certain embodiment, an anti-LOXL2 antibody or antigen binding fragment thereof comprises (i) a heavy chain variable region comprising the amino acid sequence set forth as SEQ ID NO: 37, 38, 39, 40, or 41; (ii) a light chain variable region comprising the amino acid sequence set forth as SEQ ID NO: 42, 43, 44, or 45; (iii) the complementarity determining regions (CDRs), CDR1, CDR2, and CDR3, of a heavy chain variable region comprising the amino acid sequence set forth as SEQ ID NO: 37, 38, 39, 40, or 41; and/or (iv) the CDRs, CDR1, CDR2, and CDR3, of a light chain variable region comprising the amino acid sequence set forth as SEQ ID NO: 42, 43, 44, or 45. In certain other embodiment, the anti-LOXL2 antibody or antigen binding fragment thereof comprises a heavy chain variable region comprising the amino acid sequence set forth as SEQ ID NO: 37, 38, 39, 40, or 41; and a light chain variable region comprising the amino acid sequence set forth as SEQ ID NO: 42, 43, 44, or 45. In some embodiment, AB0024 may be referred to by the sequences, wherein a heavy chain variable region comprising the amino acid sequence set forth as SEQ ID NO: 37, 38, 39, 40, or 41; and/or a light chain variable region comprising the amino acid sequence set forth as SEQ ID NO: 42, 43, 44, or 45.

In particular embodiments, an antibody according to the present disclosure not only binds to a lysyl oxidase-type enzyme but also reduces or inhibits uptake or internalization of the lysyl oxidase-type enzyme, e.g., via integrin beta 1 or other cellular receptors or proteins. Such an antibody could, for example, bind to extracellular matrix proteins, cellular receptors, and/or integrins.

Exemplary antibodies that recognize lysyl oxidase-type enzymes, and additional disclosure relating to antibodies to lysyl oxidase-type enzymes, is provided in co-owned U.S. Patent Application Publications No. US 2009/0053224 and US 2009/0104201, the disclosures of which, including all anti-LOX, anti-LOXL1, anti-LOXL2, anti-LOXL3, and anti-LOXL4 antibody sequences (including CDR, heavy chain and light chain sequences), methods of making the antibodies, and antibody variants, are herein incorporated by reference in their entireties.

### Polynucleotides Targeting LOX/LOXL

Inhibition of a lysyl oxidase-type enzyme can be effected by down-regulating expression of the lysyl oxidase enzyme at either the transcriptional or translational level. One such method of modulation involves the use of antisense oligo- or polynucleotides capable of sequence-specific binding with a mRNA transcript encoding a lysyl oxidase-type enzyme.

In particular embodiments, the polynucleotide inhibitors of the present disclosure can reduce or inhibits uptake or internalization of LOX or LOXL. It is contemplated that such a polynucleotide inhibitor could reduce EMT and thus is useful for the applications disclosed herein.

In certain embodiments, the polynucleotide inhibitors of the present disclosure can reduce or inhibit the lysyl oxidase enzymatic activity of LOX or LOXL. It is contemplated that such a polynucleotide inhibitor could reduce EMT and thus is useful for the applications disclosed herein.

### Antisense Oligonucleotides

Binding of an antisense oligonucleotide (or antisense oligonucleotide analogue) to a target mRNA molecule can lead to the enzymatic cleavage of the hybrid by intracellular RNase H. In certain cases, formation of an antisense RNA-mRNA hybrid can interfere with correct splicing. In both cases, the number of intact, functional target mRNAs, suitable for translation, is reduced or eliminated. In other cases, binding of an antisense oligonucleotide or oligonucleotide analogue to a target mRNA can prevent (e.g., by steric hindrance) ribosome binding, thereby preventing translation of the mRNA.

Antisense oligonucleotides can comprise any type of nucleotide subunit, e.g., they can be DNA, RNA, analogues such as peptide nucleic acids (PNA), or mixtures of the preceding. RNA oligonucleotides form a more stable duplex with a target mRNA molecule, but the unhybridized oligonucleotides are less stable intracellularly than other types of oligonucleotides and oligonucleotide analogues. This can be counteracted by expressing RNA oligonucleotides inside a cell using vectors designed for this purpose. This approach may be used, for example, when attempting to target a mRNA that encodes an abundant and long-lived protein.

Additional considerations can be taken into account when designing antisense oligonucleotides, including: (i) sufficient specificity in binding to the target sequence; (ii) solubility; (iii) stability against intra- and extracellular nucleases; (iv) ability to penetrate the cell membrane; and (v) when used to treat an organism, low toxicity.

Algorithms for identifying oligonucleotide sequences with the highest predicted binding affinity for their target mRNA, based on a thermodynamic cycle that accounts for the energy of structural alterations in both the target mRNA and the oligonucleotide, are available. For example, Walton et al. (1999) Biotechnol. Bioeng. 65:1-9 used such a method to design antisense oligonucleotides directed to rabbit β-globin (RBG) and mouse tumor necrosis factor-.alpha. (TNFα) transcripts. The same research group has also reported that the antisense activity of rationally selected oligonucleotides against three model target mRNAs (human lactate dehydrogenase A and B and rat gp130) in cell culture proved effective in almost all cases. This included tests against three different targets in two cell types using oligonucleotides made by both phosphodiester and phosphorothioate chemistries.

In addition, several approaches for designing and predicting efficiency of specific oligonucleotides using an in vitro system are available. See, e.g., Matveeva et al. (1998) Nature Biotechnology 16:1374-1375.

An antisense oligonucleotide according to the present disclosure includes a polynucleotide or a polynucleotide analogue of at least 10 nucleotides, for example, between 10 and 15, between 15 and 20, at least 17, at least 18, at least 19, at least 20, at least 22, at least 25, at least 30, or even at least 40 nucleotides. Such a polynucleotide or polynucleotide analogue is able to anneal or hybridize (i.e., form a double-stranded structure on the basis of base complementarity) in vivo, under physiological conditions, with a mRNA encoding a lysyl oxidase-type enzyme, e.g., LOX or LOXL2.

Antisense oligonucleotides according to the present disclosure can be expressed from a nucleic acid construct administered to a cell or tissue. Optionally, expression of the antisense sequences is controlled by an inducible promoter, such that expression of antisense sequences can be switched on and off in a cell or tissue. Alternatively antisense oligonucleotides can be chemically synthesized and administered directly to a cell or tissue, as part of, for example, a pharmaceutical composition.

Antisense technology has led to the generation of highly accurate antisense design algorithms and a wide variety of oligonucleotide delivery systems, thereby enabling those of ordinary skill in the art to design and implement antisense approaches suitable for downregulating expression of known sequences. For additional information relating to antisense technology, see, for example, Lichtenstein et al., Antisense Technology: A Practical Approach, Oxford University Press, 1998.

### Small RNA and RNAi

Another method for inhibition of the activity of a lysyl oxidase-type enzyme is RNA interference (RNAi), an approach which utilizes double-stranded small interfering RNA (siRNA) molecules that are homologous to a target mRNA and lead to its degradation. Carthew (2001) Curr. Opin. Cell. Biol. 13:244-248.

RNA interference is typically a two-step process. In the first step, which is termed as the initiation step, input dsRNA is digested into 21-23 nucleotide (nt) small interfering RNAs (siRNAs), probably by the action of Dicer, a member of the RNase III family of double-strand-specific ribonucleases, which cleaves double-stranded RNA in an ATP-dependent manner. Input RNA can be delivered, e.g., directly or via a transgene or a virus. Successive cleavage events degrade the RNA to 19-21 by duplexes (siRNA), each with 2-nucleotide 3′ overhangs. Hutvagner et al. (2002) Curr. Opin. Genet. Dev. 12:225-232; Bernstein (2001) Nature 409:363-366.

In the second, effector step, siRNA duplexes bind to a nuclease complex to form the RNA-induced silencing complex (RISC). An ATP-dependent unwinding of the siRNA duplex is required for activation of the RISC. The active RISC (containing a single siRNA and an RNase) then targets the homologous transcript by base pairing interactions and typically cleaves the mRNA into fragments of approximately 12 nucleotides, starting from the 3′ terminus of the siRNA. Hutvagner et al., supra; Hammond et al. (2001) Nat. Rev. Gen. 2:110-119; Sharp (2001) Genes. Dev. 15:485-490.

RNAi and associated methods are also described in Tuschl (2001) Chem. Biochem. 2:239-245; Cullen (2002) Nat. Immunol. 3:597-599; and Brantl (2002) Biochem. Biophys. Acta. 1575:15-25.

An exemplary strategy for synthesis of RNAi molecules suitable for use with the present disclosure, as inhibitors of the activity of a lysyl oxidase-type enzyme, is to scan the appropriate mRNA sequence downstream of the start codon for AA dinucleotide sequences. Each AA, plus the downstream (i.e., 3′ adjacent) 19 nucleotides, is recorded as a potential siRNA target site. Target sites in coding regions are preferred, since proteins that bind in untranslated regions (UTRs) of a mRNA, and/or translation initiation complexes, may interfere with binding of the siRNA endonuclease complex. Tuschl (2001) supra. It will be appreciated though, that siRNAs directed at untranslated regions can also be effective, as has been demonstrated in the case wherein siRNA directed at the 5′ UTR of the GAPDH gene mediated about 90% decrease in cellular GAPDH mRNA and completely abolished protein level (www.ambion.com/techlib/tn/91/912.html). Once a set of potential target sites is obtained, as described above, the sequences of the potential targets are compared to an appropriate genomic database (e.g., human, mouse, rat etc.) using a sequence alignment software, (such as the BLAST software available from NCBI at www.ncbi.nlm.nih.gov/BLAST/). Potential target sites that exhibit significant homology to other coding sequences are rejected.

Qualifying target sequences are selected as templates for siRNA synthesis. Selected sequences can include those with low G/C content as these have been shown to be more effective in mediating gene silencing, compared to those with G/C content higher than 55%. Several target sites can be selected along the length of the target gene for evaluation. For better evaluation of the selected siRNAs, a negative control is used in conjunction. Negative control siRNA can include a sequence with the same nucleotide composition as a test siRNA, but lacking significant homology to the genome. Thus, for example, a scrambled nucleotide sequence of the siRNA may be used, provided it does not display any significant homology to any other gene.

The siRNA molecules of the present disclosure can be transcribed from expression vectors which can facilitate stable expression of the siRNA transcripts once introduced into a host cell. These vectors are engineered to express small hairpin RNAs (shRNAs), which are processed in vivo into siRNA molecules capable of carrying out gene-specific silencing. See, for example, Brummelkamp et al. (2002) Science 296:550-553; Paddison et al (2002) Genes Dev. 16:948-958; Paul et al. (2002) Nature Biotech. 20:505-508; Yu et al. (2002) Proc. Natl. Acad. Sci. USA 99:6047-6052.

Small hairpin RNAs (shRNAs) are single-stranded polynucleotides that form a double-stranded, hairpin loop structure. The double-stranded region is formed from a first sequence that is hybridizable to a target sequence, such as a polynucleotide encoding a lysyl oxidase-type enzyme (e.g., a LOX or LOXL2 mRNA) and a second sequence that is complementary to the first sequence. The first and second sequences form a double stranded region; while the un-base-paired linker nucleotides that lie between the first and second sequences form a hairpin loop structure. The double-stranded region (stem) of the shRNA can comprise a restriction endonuclease recognition site.

A shRNA molecule can have optional nucleotide overhangs, such as 2-bp overhangs, for example, 3′ UU-overhangs. While there may be variation, stem length typically ranges from approximately 15 to 49, approximately 15 to 35, approximately 19 to 35, approximately 21 to 31 bp, or approximately 21 to 29 bp, and the size of the loop can range from approximately 4 to 30 bp, for example, about 4 to 23 bp.

For expression of shRNAs within cells, plasmid vectors can be employed that contain a promoter (e.g., the RNA Polymerase III H1-RNA promoter or the U6 RNA promoter), a cloning site for insertion of sequences encoding the shRNA, and a transcription termination signal (e.g., a stretch of 4-5 adenine-thymidine base pairs). Polymerase III promoters generally have well-defined transcriptional initiation and termination sites, and their transcripts lack poly(A) tails. The termination signal for these promoters is defined by the polythymidine tract, and the transcript is typically cleaved after the second encoded uridine. Cleavage at this position generates a 3′ UU overhang in the expressed shRNA, which is similar to the 3′ overhangs of synthetic siRNAs. Additional methods for expressing shRNA in mammalian cells are described in the references cited above.

An example of a suitable shRNA expression vector is pSUPER™ (Oligoengine, Inc., Seattle, Wash.), which includes the polymerase-III H1-RNA gene promoter with a well defined transcriptional start site and a termination signal consisting of five consecutive adenine-thymidine pairs. Brummelkamp et al., supra. The transcription product is cleaved at a site following the second uridine (of the five encoded by the termination sequence), yielding a transcript which resembles the ends of synthetic siRNAs, which also contain nucleotide overhangs. Sequences to be transcribed into shRNA are cloned into such a vector such that they will generate a transcript comprising a first sequence complementary to a portion of a mRNA target (e.g., a mRNA encoding a lysyl oxidase-type enzyme), separated by a short spacer from a second sequence comprising the reverse complement of the first sequence. The resulting transcript folds back on itself to form a stem-loop structure, which mediates RNA interference (RNAi).

Another suitable siRNA expression vector encodes sense and antisense siRNA under the regulation of separate pol III promoters. Miyagishi et al. (2002) Nature Biotech. 20:497-500. The siRNA generated by this vector also includes a five thymidine (T5) termination signal.

siRNAs, shRNAs and/or vectors encoding them can be introduced into cells by a variety of methods, e.g., lipofection. Vector-mediated methods have also been developed. For example, siRNA molecules can be delivered into cells using retroviruses. Delivery of siRNA using retroviruses can provide advantages in certain situations, since retroviral delivery can be efficient, uniform and immediately selects for stable “knock-down” cells. Devroe et al. (2002) BMC Biotechnol. 2:15.

Recent scientific publications have validated the efficacy of such short double stranded RNA molecules in inhibiting target mRNA expression and thus have clearly demonstrated the therapeutic potential of such molecules. For example, RNAi has been utilized for inhibition in cells infected with hepatitis C virus (McCaffrey et al. (2002) Nature 418:38-39), HIV-1 infected cells (Jacque et al. (2002) Nature 418:435-438), cervical cancer cells (Jiang et al. (2002) Oncogene 21:6041-6048) and leukemic cells (Wilda et al. (2002) Oncogene 21:5716-5724)

### V. COMPOSITIONS

The LOX/LOXL inhibitors or antagonists contemplated herein can be used as a composition when combined with a pharmaceutically acceptable carrier or excipient. In particular embodiments, the contemplated pharmaceutical compositions are useful for administration to a subject in vivo, in vitro, or ex vivo, and for treating, preventing or ameliorating at least one symptom associated with heart failure, idiopathic dilated cardiomyopathy (IDCM), and cardiac fibrosis.

In one embodiment, the LOX/LOXL inhibitor is a LOXL2 inhibitor.

In certain embodiments, pharmaceutical compositions are used to reduce the extent of fibrosis, myocardial remodeling, myocardial stiffness during heart failure, cardiac arrhythmias, cardiac myofibroblast activation and/or to improve systolic and diastolic heart function.

Pharmaceutically acceptable carriers are physiologically acceptable to the administered patient and retain the therapeutic properties of the antibodies or peptides with which it is administered. Pharmaceutically-acceptable carriers and their formulations are and generally described in, for example, Remington′ pharmaceutical Sciences (18.sup.th Edition, ed. A. Gennaro, Mack Publishing Co., Easton, Pa. 1990). One exemplary pharmaceutical carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antibodies or peptides from the administration site of one organ, or portion of the body, to another organ, or portion of the body. Each carrier must be “acceptable” in the sense of being compatible with the other ingredients of the formulation and not injurious to the patient. Nor should a pharmaceutically acceptable carrier alter the specific activity of the antagonists. Exemplary carriers and excipients have been provided elsewhere herein.

In one embodiment, pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration are contemplated. Pharmaceutical compositions or pharmaceutical formulations therefore refer to a composition suitable for pharmaceutical use in a subject. The pharmaceutical compositions and formulations include an amount of an invention compound, for example, an effective amount of an antagonist of the invention, and a pharmaceutically or physiologically acceptable carrier.

Pharmaceutical compositions can be formulated to be compatible with a particular route of administration, systemic or local. Thus, pharmaceutical compositions include carriers, diluents, or excipients suitable for administration by various routes.

In a further embodiment, the compositions contemplated herein comprise a pharmaceutically acceptable additive in order to improve the stability of the antagonist in composition and/or to control the release rate of the composition. Pharmaceutically acceptable additives of the present invention do not alter the specific activity of the subject antagonist. A preferable pharmaceutically acceptable additive is a sugar such as mannitol, sorbitol, glucose, xylitol, trehalose, sorbose, sucrose, galactose, dextran, dextrose, fructose, lactose and mixtures thereof. Pharmaceutically acceptable additives of the present invention can be combined with pharmaceutically acceptable carriers and/or excipients such as dextrose. In another embodiment, a preferable pharmaceutically acceptable additive is a surfactant such as polysorbate 20 or polysorbate 80 to increase stability of the peptide and decrease gelling of the pharmaceutical solution. The surfactant can be added to the composition in an amount of 0.01% to 5% of the solution. Addition of such pharmaceutically acceptable additives increases the stability and half-life of the composition in storage.

The formulation and delivery methods will generally be adapted according to the site and the disease to be treated. Exemplary formulations include, but are not limited to, those suitable for parenteral administration, e.g., intravenous, intra-arterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. The dosage of the compounds of the invention will vary according to the extent and severity of the need for treatment, the activity of the administered composition, the general health of the subject, and other considerations well known to the skilled artisan.

Formulations or enteral (oral) administration can be contained in a tablet (coated or uncoated), capsule (hard or soft), microsphere, emulsion, powder, granule, crystal, suspension, syrup or elixir. Conventional nontoxic solid carriers which include, for example, pharmaceutical grades of mannitol, lactose, starch, magnesium stearate, sodium saccharin, talcum, cellulose, glucose, sucrose, magnesium carbonate, can be used to prepare solid formulations. Supplementary active compounds (e.g., preservatives, antibacterial, antiviral and antifungal agents) can also be incorporated into the formulations. A liquid formulation can also be used for enteral administration. The carrier can be selected from various oils including petroleum, animal, vegetable or synthetic, for example, peanut oil, soybean oil, mineral oil, sesame oil. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Pharmaceutical compositions for enteral, parenteral, or transmucosal delivery include, for example, water, saline, phosphate buffered saline, Hank's solution, Ringer's solution, dextrose/saline, and glucose solutions. The formulations can contain auxiliary substances to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. Additional parenteral formulations and methods are described in Bai (1997) J. Neuroimmunol. 80:65 75; Warren (1997) J. Neurol. Sci. 152:31 38; and Tonegawa (1997) J. Exp. Med. 186:507 515. The parenteral preparation can be enclosed in ampules, disposable syringes or multiple dose vials made of glass or plastic.

Pharmaceutical compositions for intradermal or subcutaneous administration can include a sterile diluent, such as water, saline solution, fixed oils, polyethylene glycols, glycerine, propylene glycol or other synthetic solvents; antibacterial agents such as benzyl alcohol or methyl parabens; antioxidants such as ascorbic acid, glutathione or sodium bisulfite; chelating agents such as ethylenediaminetetraacetic acid; buffers such as acetates, citrates or phosphates and agents for the adjustment of tonicity such as sodium chloride or dextrose.

Pharmaceutical compositions for injection include aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as manitol, sorbitol, and sodium chloride may be included in the composition. The resulting solutions can be packaged for use as is, or lyophilized; the lyophilized preparation can later be combined with a sterile solution prior to administration.

Pharmaceutically acceptable carriers can contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (see, e.g., Fix (1996) Pharm Res. 13:1760 1764; Samanen (1996) J. Pharm. Pharmacol. 48:119 135; and U.S. Pat. No. 5,391,377, describing lipid compositions for oral delivery of therapeutic agents).

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be through nasal sprays or suppositories (see, e.g., Sayani (1996) “Systemic delivery of peptides and proteins across absorptive mucosae” Crit. Rev. Ther. Drug Carrier Syst. 13:85 184). For transdermal administration, the active compound can be formulated into ointments, salves, gels, or creams as generally known in the art. Transdermal delivery systems can also be achieved using patches.

For inhalation delivery, the pharmaceutical formulation can be administered in the form of an aerosol or mist. For aerosol administration, the formulation can be supplied in finely divided form along with a surfactant and propellant. In another embodiment, the device for delivering the formulation to respiratory tissue is in which the formulation vaporizes. Other delivery systems known in the art include dry powder aerosols, liquid delivery systems, inhalers, air jet nebulizers and propellant systems (see, e.g., Patton (1998) Biotechniques 16:141 143; Dura Pharmaceuticals, San Diego, Calif.; Aradigm, Hayward, Calif.; Aerogen, Santa Clara, Calif.; and Inhale Therapeutic Systems, San Carlos, Calif.).

Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation and Nova Pharmaceuticals, Inc. Liposomal suspensions (including liposomes targeted to cells or tissues using antibodies or viral coat proteins) can also be used as pharmaceutically acceptable carriers. These can be prepared according to methods known in the art, for example, as described in U.S. Pat. Nos. 4,235,871; 4,501,728; 4,522,811; 4,837,028; 6,110,490; 6,096,716; 5,283,185; 5,279,833; Akimaru (1995) Cytokines Mol. Ther. 1:197 210; Alving (1995) Immunol. Rev. 145:5 31; and Szoka (1980) Ann. Rev. Biophys. Bioeng. 9:467). Biodegradable microspheres or capsules or other biodegradable polymer configurations capable of sustained delivery of small molecules including peptides are known in the art (see, e.g., Putney (1998) Nat. Biotechnol. 16:153 157). Compounds of the invention can be incorporated within micelles (see, e.g., Suntres (1994) J. Pharm. Pharmacol. 46:23 28; Woodle (1992) Pharm. Res. 9:260 265). Antagonists can be attached to the surface of the lipid monolayer or bilayer. For example, antagonists can be attached to hydrazide-PEG-(distearoylphosphatidy-1) ethanolamine-containing liposomes (see, e.g., Zalipsky (1995) Bioconjug. Chem. 6: 705 708). Alternatively, any form of lipid membrane, such as a planar lipid membrane or the cell membrane of an intact cell, e.g., a red blood cell, can be used. Liposomal and lipid-containing formulations can be delivered by any means, including, for example, intravenous, transdermal (see, e.g., Vutla (1996) J. Pharm. Sci. 85:5 8), transmucosal, or oral administration.

Compositions contemplated herein can be combined with other therapeutic moieties or imaging/diagnostic moieties as provided herein. Therapeutic moieties and/or imaging moieties can be provided as a separate composition, or as a conjugated moiety. Linkers can be included for conjugated moieties as needed and have been described elsewhere herein.

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286 288 (1982) via a disulfide-interchange reaction. A chemotherapeutic agent (such as Doxorubicin) is optionally contained within the liposome. See Gabizon et al., J. National Cancer Inst., 81(19): 1484 (1989).

Lipofections or liposomes can also be used to deliver the anti-LOX antibody, or an antibody fragment, into cells. Where antibody fragments are used, the smallest inhibitory fragment that specifically binds to the binding domain of the target protein can be used. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889 7893 (1993). The formulation herein can also contain more than one active compound as necessary for the particular indication being treated, including, for example, those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended. The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles, and nanocapsules) or in macroemulsions. Such techniques are disclosed in Remington's Pharmaceutical Sciences, supra.

Formulations for in vivo administration are sterile. Sterilization can be readily accomplished via filtration through sterile filtration membranes.

Sustained-release preparations can be prepared. Suitable examples of sustained-release preparations include semipermeable matrices of solid hydrophobic polymers, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Various other pharmaceutical compositions and techniques for their preparation and use will be known to those of skill in the art in light of the present disclosure. For a detailed listing of suitable pharmacological compositions and associated administrative techniques one can refer to the detailed teachings herein, which can be further supplemented by texts such as Remington: The Science and Practice of Pharmacy 20th Ed. (Lippincott, Williams & Wilkins 2003).

Pharmaceutical compositions contemplated by the present invention have been described above. In one embodiment of the present invention, the pharmaceutical compositions are formulated to be free of pyrogens such that they are acceptable for administration to human patients. Testing pharmaceutical compositions for pyrogens and preparing pharmaceutical compositions free of pyrogens are well understood to one of ordinary skill in the art.

One embodiment of the present invention contemplates the use of any of the pharmaceutical compositions of the present invention to make a medicament for treating a disorder of the present invention. Medicaments can be formulated based on the physical characteristics of the patient/subject needing treatment, and can be formulated in single or multiple formulations based on the stage of the cancerous tissue. Medicaments of the present invention can be packaged in a suitable pharmaceutical package with appropriate labels for the distribution to hospitals and clinics wherein the label is for the indication of treating a disorder as described herein in a subject. Medicaments can be packaged as a single or multiple units. Instructions for the dosage and administration of the pharmaceutical compositions of the present invention can be included with the pharmaceutical packages and kits described below.

### IX. THERAPEUTIC METHODS

The pharmaceutical formulations contemplated herein can be used to treat, prevent, or ameliorate at least one symptom associated with a cardiovascular injury. As used herein, the terms “cardiovascular system” or “cardiovascular” refer to the heart and the network of arteries, veins, and capillaries that transport blood throughout the body. A “cardiovascular injury” is an injury to the heart, arteries, veins, or capillaries. Illustrative examples of cardiovascular injuries suitable for treating with the compositions and methods contemplated herein include, but are not limited to heart failure, e.g., diastolic heart failure and systolic heart failure; atrial fibrillation; idiopathic dilated cardiomyopathy (IDCM); and cardiac fibrosis.

In a preferred embodiment, a composition contemplated herein is administered to a subject to treat, prevent, or ameliorate at least one symptom associated with heart failure or IDCM. In some embodiment, a composition contemplated herein is administered to a subject to treat, prevent, or ameliorate at least one symptom associated with atrial fibrillation. In certain embodiments, pharmaceutical compositions are used to reduce the extent of fibrosis, myocardial remodeling, myocardial stiffness during heart failure, cardiac myofibroblast activation, and/or to improve systolic and diastolic heart function. In various embodiments, a method of reducing or decreasing the expression or enzymatic activity of LOX or LOXL in a subject having heart failure, IDCM, or cardiac fibrosis comprising administering one or more agents, e.g., anti-LOX or anti-LOXL antibodies, or small molecules or inhibitory nucleic acids directed against LOX or LOXL, contemplated herein is provided. In various embodiments, a method of reducing or decreasing the expression or enzymatic activity of LOX or LOXL in a subject having atrial fibrillation comprising administering one or more agents, e.g., anti-LOX or anti-LOXL antibodies, or small molecules or inhibitory nucleic acids directed against LOX or LOXL, contemplated herein is provided. In one embodiment, the LOX/LOXL inhibitor is a LOXL2 inhibitor. In other embodiment, provided is a method of treating, preventing, or ameliorating at least one symptom associated with heart failure, IDCM, cardiac fibrosis, or atrial fibrillation to a subject in need thereof administering a therapeutically effectively amount of an anti-LOXL2 antibody or antigen binding fragment thereof. In some other embodiment, the anti-LOXL2 antibody or antigen binding fragment thereof comprises (i) a heavy chain variable region comprising the amino acid sequence set forth as SEQ ID NO: 37, 38, 39, 40, or 41; (ii) a light chain variable region comprising the amino acid sequence set forth as SEQ ID NO: 42, 43, 44, or 45; (iii) the complementarity determining regions (CDRs), CDR1, CDR2, and CDR3, of a heavy chain variable region comprising the amino acid sequence set forth as SEQ ID NO: 37, 38, 39, 40, or 41; and/or (iv) the CDRs, CDR1, CDR2, and CDR3, of a light chain variable region comprising the amino acid sequence set forth as SEQ ID NO: 42, 43, 44, or 45. In certain other embodiment, the anti-LOXL2 antibody or antigen binding fragment thereof comprises a heavy chain variable region comprising the amino acid sequence set forth as SEQ ID NO: 37, 38, 39, 40, or 41; and a light chain variable region comprising the amino acid sequence set forth as SEQ ID NO: 42, 43, 44, or 45.

Inhibition of LOX or LOXL can have one or more effects in a subject such as, for example, reducing the extent of fibrosis, reducing myocardial remodeling, reducing myocardial stiffness during heart failure, reducing cardiac myofibroblast activation, and/or improving systolic and diastolic heart function. In one embodiment, the LOX/LOXL inhibitor is a LOXL2 inhibitor.

Pharmaceutical compositions of the present invention are administered in therapeutically effective amounts which are effective for producing some desired therapeutic effect at a reasonable benefit/risk ratio applicable to any medical treatment. For the administration of the present pharmaceutical compositions to human patients, the pharmaceutical compositions of the present invention can be formulated by methodology known by one of ordinary skill in the art to be substantially free of pyrogens such that they do not induce an inflammatory response.

The terms “treating,” “treatment”, and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any treatment of a disease in a mammal, and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. The expected progression-free survival times can be measured in months to years, depending on prognostic factors including the number of relapses, stage of disease, and other factors. Prolonging survival includes without limitation times of at least 1 month (mo), about at least 2 months (mos.), about at least 3 mos., about at least 4 mos., about at least 6 mos., about at least 1 year, about at least 2 years, about at least 3 years, or more. Overall survival can also be measured in months to years. The patient's symptoms can remain static or can decrease.

As used herein, the phrase “ameliorating at least one symptom of” refers to decreasing one or more symptoms of the disease or condition for which the subject is being treated. In particular embodiments, the disease or condition being treated is heart failure, atrial fibrillation, idiopathic dilated cardiomyopathy (IDCM), and cardiac fibrosis, wherein the one or more symptoms ameliorated include, but are not limited to, reducing the extent of fibrosis, reducing myocardial remodeling, reducing myocardial stiffness during heart failure, reducing cardiac myofibroblast activation, and/or improving systolic and diastolic heart function.

As used herein, the term “amount” refers to “an amount effective” or “an effective amount” of cells sufficient to achieve a beneficial or desired prophylactic or therapeutic result, including clinical results. In one embodiment an effect amount refers to the amount of a therapeutic agent sufficient to prevent, ameliorate one symptom of, or treat a disease contemplated herein.

As used herein, the term “therapeutically effective amount” or “effective amount” refers to an amount of a therapeutic agent that when administered alone or in combination with another therapeutic agent to a cell, tissue, or subject is effective to prevent or ameliorate the disease condition or the progression of the disease. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. For example, when in vivo administration of an anti-LOX/anti-LOXL2 antibody is employed, normal dosage amounts can vary from about 10 ng/kg to up to 100 mg/kg of mammal body weight or more per day, preferably about 1 μg/kg/day to 50 mg/kg/day, optionally about 100 μg/kg/day to 20 mg/kg/day, 500 μg/kg/day to 10 mg/kg/day, or 1 mg/kg/day to 10 mg/kg/day, depending upon the route of administration.

A physician or veterinarian having ordinary skill in the art can readily determine and prescribe the effective amount (ED50) of the pharmaceutical composition required. For example, the physician or veterinarian can start doses of the compounds of the invention employed in the pharmaceutical composition at levels lower than that required in order to achieve the desired therapeutic effect and gradually increase the dosage until the desired effect is achieved.

As used herein, the term “subject” means mammalian subjects. Exemplary subjects include, but are not limited to humans, monkeys, dogs, cats, mice, rats, cows, horses, goats and sheep. In some embodiments, the subject has heart disease or cardiovascular injury and can be treated with the agent of the present invention as described below. In some other embodiments, the subject has atrial fibrillation can be treated with the agent of the present invention as described below. The terms “subject in need thereof” or “patient in need thereof” refer to a subject or a patient who may have, is diagnosed, or is suspected to have diseases, or disorders, or conditions that would benefit from the treatment described herein. In certain embodiments, the subject or patient who (i) has not received any treatment, (ii) has received prior treatment and is not responsive or did not exhibit improvement, or (iii) is relapse or resistance to prior treatment.

Regardless of the route of administration selected, the compounds of the present invention, which are used in a suitably hydrated form, and/or the pharmaceutical compositions of the present invention are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

In one embodiment, administration of a therapeutic agent contemplated herein results in an improvement the subject's condition. In another aspect, administration of the antibodies prevents the subject's condition from worsening and/or prolongs survival of the patient.

The patient can be a mammal such as a human or a non-human. Such a patient can be symptomatic or asymptomatic.

Compositions can be administered locally, regionally or systemically by any suitable route provided herein.

Also provided herein are methods, compositions, and kits for treating or preventing a disease associated with heart failure, IDCM, cardiac arrhythmia, or cardiac fibrosis in a subject, comprising: administering to the subject an effective amount of an inhibitor of LOX or LOXL. Also provided herein are methods, compositions, and kits for treating or preventing a disease associated with AF in a subject, comprising: administering to the subject an effective amount of an inhibitor of LOX or LOXL.

In one embodiment, one or more symptoms of the patient are ameliorated. Amelioration can be manifested as, for example, reduction in pain, inhibition of fibrosis, reducing myocardial remodeling, reducing myocardial stiffness during heart failure, reducing cardiac myofibroblast activation, and/or improving systolic and diastolic heart function.

The inhibitor of LOX or LOXL may be an inhibitor of an active LOX or LOXL. The active LOX or LOXL may be a mature form of the LOX or LOXL after proteolytic processing or cleavage. Examples of LOXL include but are not limited to LOXL1, LOXL2, LOXL3, and LOXL4. The inhibitor LOX or LOXL can be an inhibitor of active LOX, LOXL2 or LOXL4. In some embodiments, the inhibitor LOX or LOXL inhibits both active LOX and active LOXL2.

The LOX or LOXL inhibitor may be an antibody against LOX or LOXL, a small molecule inhibitor, siRNA, shRNA or an antisense polynucleotide against LOX or LOXL.

Expression of specific lysyl oxidases may be associated with different stages of the inflammatory response and wound healing after heart failure, IDCM, cardiac arrhythmia, e.g., AF, or cardiac fibrosis. By specifically inhibiting the particular lysyl oxidase/s associated with the downstream fibrotic response, the detrimental consequences of cardiac remodeling and wound healing can be avoided, while allowing the immediate post-injury repair/healing process to occur.

The post-injury healing response can induce expression of LOX/LOXL but if this process continues unchecked, excessive cross-linking leads to extracellular matrix myocardial remodeling or cardiac fibrosis that results in cardiac dysfunction. The enzymes that break down matrices and cross-linked collagen or elastin appear to function more slowly or less efficiently and are outpaced by crosslinking events. As LOX/LOXL also plays a role in epithelial-mesenchymal transition (EMT), this contributes further to cardiomyocyte remodeling and cardiomyocyte hypertrophy, in addition to matrix remodeling.

In one embodiment, anti-LOX/LOXL treatment may be initiated 2, 4, 6, 8, 10, 12, 14, 16, 16, 20, 22, 24, 36, 48 or more hours after the cardiac insult or diagnosis thereof, inclusive of all integers and times in between. Additionally, anti-LOX/LOXL treatment may be initiated 2, 3, 4, 5, 6, 7, 8, 9, 10, 11, 12, 13, 14, or more days after the cardiac insult or diagnosis thereof. Similarly, increases in blood pressure (hypertension) result in increased collagen deposition and reduced protein degradation in cardiac tissue. (Berk et al., J. Clin. Invest., 117(3): 568-575 (2007)). Anti-LOX/LOXL treatment initiated following diagnosis and/or establishment of heart failure, IDCM, or cardiac fibrosis can prevent, reduce, or ameliorate myocardial remodeling, myocardial stiffness during heart failure, cardiac myofibroblast activation, and/or improving systolic and diastolic heart function. Such anti-LOX/LOXL treatment is initiated 2, 3, 4, 5, 6, 7, 8, 9, 10, 11, 12, 13, 14 or more days after one or more symptoms associated with heart failure, IDCM, or cardiac fibrosis are diagnosed or detected.

In some embodiments, biomarkers may be used to determine when an inappropriate level of cross-linking might be occurring: LOX levels have been shown to correlate with C reactive protein (CRP), a commonly used biomarker, and treatment may begin when CRP levels are elevated above appropriate normal levels. More directly, methods and test kits exist to measure the release of cross-linked collagen telopeptides in urine or blood. Elevated levels of these collagen fragments may indicate a transition from reparative fibrosis to reactive (mal-adaptive) fibrosis. In addition, measures of cardiac function and output, including those associated with efficient contraction of the ventricle, may be made.

In some embodiments, a limited duration of treatment is envisioned. Treatment should typically be sustained only long enough to prevent or attenuate reactive fibrosis to prevent or reduce one or more symptoms associated with heart failure, IDCM, cardiac arrhythmia or cardiac fibrosis. For example, short-lived Fab antibody fragments are used when shorter durations of treatment are desired. Alternatively, full-length antibodies that have a longer half-life in serum may be used, with limited dosing over 1, 2, 3, 4, 5, 6, 7, 8, 9, 10, 11, 12, or more weeks, inclusive of all days in between. Standard tests of cardiac function may be used to monitor progress and adjust dosing as necessary, along with assessment of relevant biomarkers discussed above. Limited duration of treatment adds to the safety of this approach.

In addition to the use of therapeutic agents that inhibit expression and/or activity of LOX or LOXL enzymes, combination therapies comprising a therapeutic agent and an anti-fibrotic agent are also contemplated.

In one embodiment, a method of preventing, treating, or ameliorating one or more symptoms associated with heart failure, IDCM, cardiac arrhythmia, or cardiac fibrosis comprises administration of anti-LOX or anti-LOXL2 antibody or inhibitory nucleic acid that hybridizes to LOX or LOXL2 and an anti-fibrotic agent.

Exemplary anti-fibrotic agents include, but are not limited to the compounds such as β-aminoproprionitrile (BAPN), as well as the compounds disclosed in U.S. Pat. No. 4,965,288 to Palfreyman, et al., issued Oct. 23, 1990, entitled “Inhibitors of lysyl oxidase,” relating to inhibitors of lysyl oxidase and their use in the treatment of diseases and conditions associated with the abnormal deposition of collagen; U.S. Pat. No. 4,997,854 to Kagan, et al., issued Mar. 5, 1991, entitled “Anti-fibrotic agents and methods for inhibiting the activity of lysyl oxidase in situ using adjacently positioned diamine analogue substrate,” relating to compounds which inhibit LOX for the treatment of various pathological fibrotic states, which are herein incorporated by reference. Further exemplary inhibitors are described in U.S. Pat. No. 4,943,593 to Palfreyman, et al., issued Jul. 24, 1990, entitled “Inhibitors of lysyl oxidase,” relating to compounds such as 2-isobutyl-3-fluoro-, chloro-, or bromo-allylamine; as well as, e.g., U.S. Pat. No. 5,021,456; U.S. Pat. No. 5,5059,714; U.S. Pat. No. 5,120,764; U.S. Pat. No. 5,182,297; U.S. Pat. No. 5,252,608 (relating to 2-(1-naphthyloxymethyl)-3-fluoroallylamine); and U.S. Patent Application No. 2004/0248871, which are herein incorporated by reference. Exemplary anti-fibrotic agents also include the primary amines reacting with the carbonyl group of the active site of the lysyl oxidases, and more particularly those which produce, after binding with the carbonyl, a product stabilized by resonance, such as the following primary amines: ethylenediamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone. In another embodiment, the anti-fibrotic agents are copper chelating agents, penetrating or not penetrating the cells. Additional exemplary compounds include indirect inhibitors such compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphinate, 2-acetamidoethyl-2-acetamidoethanethiol sulphanate, sodium-4-mercaptobutanesulphinate trihydrate.

The methods contemplated herein can be performed on cells in culture, e.g., in vitro or ex vivo, or can be performed on cells present in a subject, e.g., as part of an in vivo therapeutic protocol. The therapeutic regimen can be carried out on a human or on other animal subjects. The anti-LOX or anti-LOX2 antibodies or inhibitory nucleic acids contemplated herein can be administered in any order relative to the anti-fibrotic agent. Sometimes, the inhibitory LOX/LOX2 agent and the anti-fibrotic agent and the agent are administered simultaneously or sequentially. They can be administered at different sites and on different dosage regimens. The enhanced therapeutic effectiveness of the combination therapy of the contemplated herein represents a promising alternative to conventional highly toxic regimens of anti-fibrotic agents.

### X. DIAGNOSTIC METHODS

The present disclosure also provides methods for diagnosing, monitoring, staging or detecting the diseases described above by using agents that recognize different forms of LOXL2. For example, as described above, antibodies against different forms of LOXL2 the preproprotein, secreted, mature form, can be used for these purposes.

As described above, mature LOXL2 is cleaved and can be detected by virtue of it changes in molecular weight (immunoblot) or by use of antibodies that detect the uncleaved vs. cleaved form of LOXL2, along with cellular localization by using various detection methods such as immunohistochemistry (IHC).

Samples from individuals having at least one symptom associated with heart failure and/or other cardiovascular diseases can be collected and analyzed by determining inactive or active LOXL2 levels or different forms of LOX/LOXL levels. In particular embodiments, samples from a subject that has heart failure, atrial fibrillation or IDCM can be collected and analyzed by determining inactive or active LOXL2 levels or different forms of LOX/LOXL levels. The analysis may be performed prior to the initiation of treatment using lysyl oxidase-specific therapy. Such diagnosis analysis can be performed using any sample, including but not limited to cells, protein or membrane extracts of cells, biological fluids such as sputum, blood, serum, plasma, or urine, or biological samples such as tissue samples, formalin-fixed or frozen tissue sections.

Any suitable method for detection and analysis of inactive and/or active LOXL2 can be employed. As used herein, the term “sample” refers to a sample from a human, animal, or to a research sample, e.g., a cell, tissue, organ, fluid, gas, aerosol, slurry, colloid, or coagulated material. The sample may be tested in vivo, e.g., without removal from the human or animal, or it may be tested in vitro. The sample may be tested after processing, I., by histological methods. The term “sample” may also refer to a cell, tissue, organ, or fluid that is freshly taken from a human or animal, or to a cell, tissue, organ, or fluid that is processed or stored.

In one embodiment, methods are provided for diagnosing heart failure or atrial fibrillation in a subject suspected of having a cardiovascular injury, comprising assessing active LOXL2 levels or activity in the serum of the subject, whereby an increase in active LOXL2 levels or activity in the serum in comparison with a reference sample, indicates that a subject has heart failure or atrial fibrillation.

In one embodiment, methods are provided for monitoring heart failure or atrial fibrillation in a subject that has been diagnosed as having a cardiovascular injury, comprising assessing active LOXL2 levels or activity in the serum, whereby an increase in active LOXL2 levels or activity in the serum of the subject in comparison with a reference sample, indicates that the heart failure or atrial fibrillation is worsening. In contrast, decreased LOXL2 levels or activity in the serum of the subject in comparison with a reference sample, indicates that the heart failure or atrial fibrillation is improving.

In some embodiments, the monitoring may be performed to assess the patient's response to an anti-LOXL2 treatment regimen.

The reference sample may derive from the same subject, taken from the same tumor at a different time point or from other site of the body, or from another individual.

Measurement of active LOXL2 levels may take the form of an immunological assay, which detects the presence of active LOXL2 protein with an antibody to the protein, for example, an antibody specifically binding to active or secreted LOXL2

Immunoassays also can be used in conjunction with laser induced fluorescence (see, for example, Schmalzing and Nashabeh, Electrophoresis 18:2184-93 (1997)); Bao, J. Chromatogr. B. Biomed. Sci. 699:463-80 (1997), each of which is incorporated herein by reference). Liposome immunoassays, such as flow-injection liposome immunoassays and liposome immunosensors (Rongen et al., J. Immunol. Methods 204:105-133 (1997), also can be used to determine active LOX or LOXL levels according to a method of the disclosure). Immunoassays, such as enzyme-linked immunosorbent assays (ELISAs), are useful in the methods provided herein. A radioimmunoassay also can be useful for determining whether a sample is positive for active LOXL2 or for determining the level of active LOXL2. A radioimmunoassay using, for example, an iodine-125 labeled secondary antibody, may be used.

In addition, one may measure the activity of active LOXL2, thus ignoring the amount of inactive enzyme. Enzymatic activity of active LOXL2 may be measured in a number of ways, using a soluble elastin or soluble collagen with labeled lysine as a substrate. Details of an activity assay are given in Royce et al., Biochem J. 1982 Feb. 15; 202(2): 369-371. Chromogenic assays may be used. One is described in Palamakumbura, et al. Anal Biochem. 2002 Jan. 15; 300(2):245-51.

All publications, patent applications, and issued patents cited in this specification are herein incorporated by reference as if each individual publication, patent application, or issued patent were specifically and individually indicated to be incorporated by reference in its entirety.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

This study characterized the effects of anti-LOXL2 antibodies on cardiac fibrosis and myocardial remodeling. Transaortic constriction (TAC) was used to pressure overload the heart to induce heart failure (HF). The pressure load caused was verified by the pressure gradient (>30 mmHg) across the aortic constriction using echocardiography. Two weeks after the surgical procedure with either TAC or sham, the mice were administered intraperitoneally with either anti-IgG1 or anti-LOXL2 antibody AB0023 (30 mg/kg, twice a week). Ten mice were used in each group: sham/IgG1, sham/AB0023, TAC/IgG1, and TAC/AB0023. Each group (n=10) were separated into two subgroups (n=5) for surgeries conducted one week apart.

The heart function was monitored using echocardiography every two-week and measured by catheterization in vivo at ten weeks after TAC. At the end of the study, pressure-volume loop data was collected, and the left ventricle tissues, atria and blood/serum were collected. The ventricle samples were weighed to calculate ventricle/body weight ratio for hypertrophy and characterized the levels of LOXL2, collagen I, α-smooth muscle actin-markers (aSMA), and cardiac fibrosis. Additionally, the atria samples were collected to characterize the levels of LOXL2, collagen I, aSMA and other fibrotic genes. Blood was collected and allowed to clot at room temperature. Serum was separated by 3000 rpm (Beckman 6r centrifuge) at 4° c. for 10 minutes and used for biomarker assays.

To characterize the cardiac function and the chamber size of the tested subjects, echocardiography was used to measure fractional shortening and end-systolic/end-diastolic chamber diameter. Also, cardiac catheterization and PV loop were used to measure ejection fraction, chamber size, left ventricular pressure, cardiac output, contractility, and parameters of cardiac stiffness Immunohistochemistry or qPCR were used to examine the levels of LOXL2, collagen I, αSMA, or other fibrotic genes. Also, trichrome staining was used to measure cardiac fibrosis and the collagen assay was used to measure total, soluble, and insoluble collagens. The plasma levels of BNP, TIMP-1, IL6, PICP, or TGFβ were determined using ELISA.

Results from echocardiograph showed that the anti-LOXL2 antibody reduced the progression of cardiac dysfunction induced by TAC. The effects were observed within 2 weeks of the treatment. Also, at the end of the study (10 weeks after TAC), the mice treated with AB0023 had similar levels of left ventricular fractional shortening as those at two weeks after TAC. Ten weeks after TAC, the mice treated with IgG1 developed severe cardiac hypertrophy with 81% increase of ventricle/body-weight ratio, 88% increase of end-systolic LV internal diameter (LVIDs), 39% increase of end-diastolic LV internal diameter (LVIDd), and 49% reduction of left ventricular fractional shortening. In contrast, the mice treated with AB0023 developed much less cardiac dysfunction. Compared to the IgG1-treated group, the anti-LOXL2-treated group showed a 13% decrease of ventricle/body-weight ratio (p=0.059), a 51% increase of left ventricular fractional shortening (p<0.01), and a decrease of LVIDd and LVIDs by 13% (p<0.05) and 25% (p<0.05), respectively. This suggests that the LOXL2 antibody treatment protects the mice from the heart failure induced by TAC.

Furthermore, the mechanical properties of the heart were measured by in vivo catheterization. The TAC mice treated with AB0023 had increased ejection fraction (EF) by 107% (p<0.01), stroke volume (SV) by 73% (p=0.01), stroke work (SW) by 48% (p=0.01), and cardiac output (CO) by 70% (p=0.01). Also, the TAC mice treated with AB0023 exhibited reduced end diastolic pressure (EDP) by 48% (p<0.01), end systolic volume (ESV) by 43% (p<0.001), end diastolic volume (EDV) by 19% (p<0.01), and Tau by 42% (p=0.01). Furthermore, the levels of diastolic parameters (EDP, Tau, and diastolic dp/dt) and serum biomarkers (BNP and TIMP) were normalized by AB0023. These results suggest that the LOXL2 antibody treatment improves both left ventricular systolic/diastolic function and provides therapeutic effects on both systolic and diastolic failure of the heart.

In other studies, the levels of LOXL2 in HF patients with idiopathic dilated cardiomyopathy (IDCM) were examined Immunohistochemistry and qPCR were used to determine LOXL2, collagen I, and collagen III expression in left ventricular (LV) samples from IDCM patients. In IDCM human and the TAC mice, the levels of mRNA and protein for LOXL2, collagen I, and collagen III were increased compared to the controls.

In affected myocardium of left ventricular (LV) samples from IDCM patients, LOXL2 expression was detected between cardiomyocytes and localized to fibrobroblasts as determined by DDR2 co-immunofluorescent staining. Serial sections evaluated for collagen I expression also showed the association with collagen I positive fibroblasts and extracellular matrix in the areas corresponding to LOXL2 staining. Similarly, affected myocardial tissue from the TAC mice had increased LOXL2 and collagen I expression relative to controls. Using qRT-PCR, increased mRNA levels of LOXL2 (2-fold) and collagen I (4-fold) in the TAC myocardial tissue were also detected relative to control myocardial tissue. Taken together, these results illustrate LOXL2 expression in human IDCM and the TAC murine model of cardiomyopathy.

In TAC mice, the increase in LOXL2 levels was associated with an increase of total and cross-linked collagens, perivascular and interstitial fibrosis, cardiac hypertrophy, as well as severity of systolic and diastolic dysfunction. Results showed that the group treated with ani-LOXL2 antibody AB0023 exhibited reduced cardiac hypertrophy, improved the ejection fraction and cardiac contractility, eliminated diastolic dysfunction, and abolished LV dilation. This shows the LOXL2 antibody treatment results in normalized stroke work and cardiac output in pressure-overloaded hearts, normalized cardiac diastolic parameters (end diastolic pressure and LV relaxation time constant) and plasma biomarkers (BNP and TIMP-1). Taken together, the levels of LOXL2 and collagen in the heart are upregulated in human IDCM and TAC mice. The treatment with anti-LOXL2 antibodies reduces myocardial remodeling and improves both systolic and diastolic heart function in TAC mice, suggesting that LOXL2 inhibition provides a potential therapy for HF.

### Example 2

Heart failure is associated with increased extracellular matrix (ECM) remodeling, marked myocardial fibrosis, and increased myocardial stiffness. Lysyl oxidase-like 2 (LOXL2) catalyzes oxidative deamination of the lysine or hydroxylysine residues of collagen, leading to collagen cross-linking and myocardial stiffness. The purpose of this experiment was to determine the role of LOXL2 in the activation of cardiac myofibroblasts associated with the development of myocardial fibrosis.

RNA Interference-mediated knockdown of LOXL2 in human primary cardiac fibroblasts reduced the production of TGF-β2, but not TGF-β1 or TGF-β3, in culture medium as measured by multiplex immunoassays. Knockdown of LOXL2 also led to compromised TGF-β signaling evidenced by a reduction of Smad phosphorylation and down-regulation of TGF-β-controlled gene expression, which included collagen I and αSMA of ECM synthesis and myofibroblast activation. Consistent with the loss-of-function studies, overexpression of LOXL2 in cardiac fibroblasts increased the production of TGF-β2, but not TGF-β1 or TGF-β3. Further analyses revealed that LOXL2 activated signaling to enhance production of TGF-β2, as evidenced by increased phosphorylation of AKT, 4E-BP1 and p70s6k.

These results show that LOXL2 activated cardiac myofibroblasts and ECM synthesis by sustaining TGF-β2 signaling of fibroblasts. Such LOXL2-sustained TGF-β2 signaling contributed to the persistent activation of myofibroblasts, which occurs in the development of cardiac fibrosis.

### Example 3

Serum samples from patients with heart failure and atrial fibrillation and corresponding control samples were assayed for LOXL2 protein expression (Vitek Immuno Diagnostic Assay System).

Patient serum samples were aliquoted to the assay strip. In an automated fashion, the solid phase receptor (SPR) captured LOXL2 in a sample by a specific antibody immobilized onto the SPR. Following capture and wash steps, an anti-LOXL2 detection antibody conjugated to alkaline phosphatase bound and formed a sandwich. A substrate reagent was then added to initiate a fluorescent reaction detected by the instrument. The levels of LOXL2 in the sample were correlated to the amount of relative fluorescent units that were detected. Two LOXL2 assay devices were developed.

The following samples were collected from systolic heart failure (SHF) patients exhibiting Class II-IV heart failure symptoms with ejection fraction <35%; diastolic heart failure (DHF) patients exhibiting Class II-IV heart failure symptoms with ejection fraction >50%; and permanent atrial fibrillation (AF) patients, refractory to anti-arrhythmic, cardioversion, or RF ablation therapy, and exhibiting chronic AF for greater than 1 year.

Samples did not include patents who had any of the following diseases: IPF, liver diseases (hepatitis, fatty liver diseases etc.), cancer, scleroderma, or renal failure. In addition, patients with heart failure were excluded from the permanent AF and control collections (CON) while patients showing permanent AF were excluded from the SHF, DHF, and control collections.

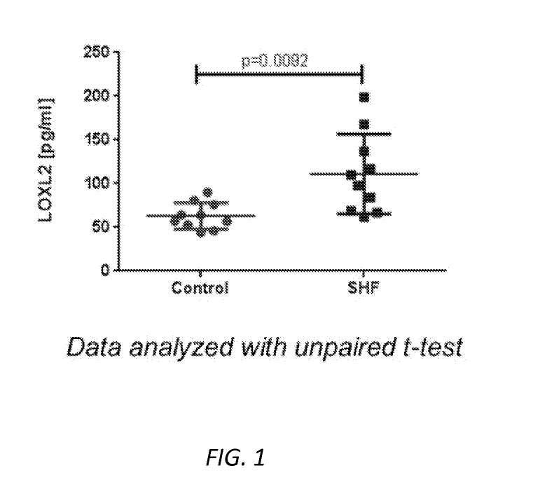

The results were summarized in Table 1 and FIG. 1. The results showed that increased LOXL2 levels in serum in permanent (PERM) AF, DHF, and SHF patient samples (Table 1, FIG. 1). Therefore, patients with heart failure or permanent AF display increased levels of LOXL2 protein in their serum, suggesting that LOXL2 may be suitable as a biomarker in various heart disease conditions.

TABLE 1
Levels of LOXL2 in serum in DHF, SHF, and AF patient samples.

| | DHF | DHF | DHF | DHF | | | PERM | | PERM | |
|---|---|---|---|---|---|---|---|---|---|---|
| | CON | Collection | CON | Collection | SHF | | AF | PERM | AF | PERM |
| | #1 | #1 | #2 | #2 | CON | SHF | CON | AF | CON\hat{ } | AF\hat{ } |
| LOXL2 | 66.6 | 78.4 | 110.9 | 174.3 | 63.2 | 111.1 | 56.6 | 67.3 | 296 | 377.3 |
| [pg/ml] | | | | | | | | | | |
| Standard | 20.1 | 18.5 | 53.6 | 95.3 | 15.1 | 45.6 | 14.1 | 25.7 | 84.3 | 101.4 |
| Deviation | | | | | | | | | | |
| Sample No. | 10 | 10 | 19 | 8 | 10 | 10 | 8 | 8 | 8 | 8 |
| p value | | 0.19 | | 0.11 | | 0.0092 | | 0.33 | | 0.1 |

\hat{ }Samples screened in the first version of the assay device

### Example 4

Gene expression levels of LOX family and BNP in the left ventricle (LV) of controls and SHF patients were determined using real-time RT-PCR.

LOXL2 gene expression was significantly increased in LV of SHF patients up to an average of 3.8±0.6 fold relative to controls. In contrast, the expression levels of other LOX family members, LOX, LOXL1, LOXL3 and LOXL4 in LV of SHF were not significantly different compared to controls. The expression level of LOXL2 significantly correlated with expression level of BNP, a heart failure biomarker (r=0.56, p=0.01) in all samples.

Plasma concentrations of NT-proBNP, ST-2 and TIMP-1 in SHF patients and controls were measured using ELISA. Serum LOXL2 was measured as described above.

Plasma concentrations of NT-pro-BNP, a heart failure biomarker and TIMP-1, a fibrotic mediator were significantly increased in SHF patients compared to controls. There were significant positive correlations between serum LOXL2 and NT-pro-BNP, LOXL2 and TIMP-1, and LOXL2 and ST-2, another HF biomarker (r=0.5, 0.6, and 0.6 respectively; p<0.05).

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for treating, preventing, or ameliorating at least one symptom associated with a heart disease or condition, comprising: administering to a subject an effective amount of an inhibitor of active lysyl oxidase or lysyl oxidase-like protein.

2. The method of claim 1, wherein the heart disease or condition is selected from the group consisting of heart failure, heart failure with preserved ejection fraction (HFpEF), heart failure with reduced ejection fraction (HFrEF), a cardiac arrhythmia and idiopathic dilated cardiomyopathy (IDCM), cardiac fibrosis, atrial fibrillation (AF), or a cardiovascular injury caused by IDCM, HFpEF, HFrEF, a cardiac arrhythmia, and cardiac fibrosis.

3. The method of claim 1, wherein ameliorating the one or more symptoms comprises reducing the extent of fibrosis, reducing myocardial remodeling, reducing myocardial stiffness during heart failure, reducing cardiac myofibroblast activation and/or improving systolic and diastolic heart function.

4. The method of claim 1, wherein the LOX or LOXL inhibitor is an antibody against LOX or LOXL, a small molecule inhibitor, siRNA, shRNA or an antisense polynucleotide against LOX or LOXL.

5. The method of claim 1, wherein the LOX or LOXL inhibitor is an antibody that specifically binds to a region of LOX or LOXL having an amino acid sequence selected from SEQ ID NOs:1-22.

6. The method of claim 1, wherein the LOX or LOXL inhibitor is parenterally administered to the subject.

7. The method of claim 1, wherein the LOX or LOXL inhibitor is administered locally to a site of cardiovascular injury.

8. The method of claim 7, wherein the LOX or LOXL inhibitor is administered via a stent.

9. The method of claim 8, wherein the LOX or LOXL inhibitor is coated on the stent.

10. The method of claim 1, wherein the LOX or LOXL inhibitor is administered locally to a site of cardiovascular injury via a catheter.

11. The method of claim 1, wherein the LOX or LOXL inhibitor is administered prior to the onset or diagnosis of the cardiovascular injury.

12. The method of claim 1, wherein the LOX or LOXL inhibitor is administered after the onset or diagnosis of the cardiovascular injury.

13. The method of claim 1, wherein the inhibitor or anti-LOXL2 antibody or antigen binding fragment thereof, comprises a heavy chain variable region comprising the amino acid sequence set forth as SEQ ID NO: 37, 38, 39, 40, or 41, and/or a light chain variable region comprising the amino acid sequence set forth as SEQ ID NO: 42, 43, 44, or 45.

14. The method of claim 1, wherein the LOXL2 inhibitor or the anti-LOXL2 antibody or antigen binding fragment thereof, comprises the complementarity determining regions (CDRs), CDR1, CDR2, and CDR3, of a heavy chain variable region comprising the amino acid sequence set forth as SEQ ID NO: 37, 38, 39, 40, or 41, and the CDRs, CDR1, CDR2, and CDR3, of a light chain variable region comprising the amino acid sequence set forth as SEQ ID NO: 42, 43, 44, or 45.

15. The method of claim 1, wherein the LOXL2 inhibitor or the anti-LOXL2 antibody or antigen binding fragment thereof, comprises a heavy chain variable region comprises the CDR1-3 amino acid sequences set forth in SEQ ID NOs: 46-48.

16. The method of claim 1, wherein the LOXL2 inhibitor or the anti-LOXL2 antibody or antigen binding fragment thereof, comprises a light chain variable region comprises the CDR1-3 amino acid sequences set forth in SEQ ID NOs: 49-51.

17. An inhibitor of active lysyl oxidase or lysyl oxidase-like protein for use in treating, preventing, or ameliorating at least one symptom associated with a cardiovascular injury selected from the group consisting of: idiopathic dilated cardiomyopathy (IDCM), heart failure, atrial fibrillation, and cardiac fibrosis.

18. A composition comprising an inhibitor of lysyl oxidase, an inhibitor of a lysyl oxidase-like protein and a pharmaceutically acceptable carrier for use in treating, preventing, or ameliorating at least one symptom associated with a cardiovascular injury selected from the group consisting of: idiopathic dilated cardiomyopathy (IDCM), heart failure, atrial fibrillation, and cardiac fibrosis.

19. A method for diagnosing heart failure or atrial fibrillation in a subject, comprising:
contacting a serum sample obtained from an individual with an anti-LOXL2 antibody;
detecting the binding of the anti-LOXL2 antibody to an anti-LOXL2 antibody/LOXL2 complex;
wherein an increase in the level of an anti-LOXL2 antibody/LOXL2 complex compared to a reference sample indicates the presence of heart failure or atrial fibrillation in the subject.

20. The method of claim 19, wherein the subject is suspected of having heart failure.

21. The method of claim 20, wherein the heart failure is diastolic heart failure.

22. The method of claim 20, wherein the heart failure is systolic heart failure.

23. The method of claim 16, wherein the subject is suspected of having atrial fibrillation

24. A method for monitoring heart failure or atrial fibrillation in a subject, comprising:
contacting a serum sample obtained from an individual with an anti-LOXL2 antibody;
detecting the binding of the anti-LOXL2 antibody to an anti-LOXL2 antibody/LOXL2 complex;
wherein an increase in the level of an anti-LOXL2 antibody/LOXL2 complex compared to a reference sample indicates a worsening of heart failure or atrial fibrillation in the subject or
wherein an decrease in the level of an anti-LOXL2 antibody/LOXL2 complex compared to a reference sample indicates an improvement of heart failure or atrial fibrillation in the subject.

25. The method of claim 24, wherein the binding of the anti-LOXL2 antibody to the anti-LOXL2 antibody/LOXL2 complex is detected by enzyme-linked immunosorbent assays (ELISA).
